(19) United States
(12) Patent Application Publication
(10) Pub. No.: US 2020/0157637 A1
(43) Pub. Date: May 21, 2020

(54) TARGETING OF ANAPLASTIC LYMPHOMA KINASE IN SQUAMOUS CELL CARCINOMA

(71) Applicant: BOARD OF REGENTS, THE UNIVERSITY OF TEXAS SYSTEM, Austin, TX (US)

(72) Inventors: Cara B. GONZALES, San Antonio, TX (US); Nameer KIRMA, San Antonio, TX (US)

(73) Assignee: BOARD OF REGENTS, THE UNIVERSITY OF TEXAS SYSTEM, Austin, TX (US)

(21) Appl. No.: 16/615,234

(22) PCT Filed: May 23, 2018
(86) PCT No.: PCT/US18/34059

§ 371 Date: Nov. 20, 2019

Related U.S. Application Data
(60) Provisional application No. 62/511,704, filed on May 26, 2017.

(51) Int. Cl. C12Q 1/6886; A61K 31/506; A61K 31/5377

(52) U.S. Cl. CPC C12Q 1/6886; A61K 31/506; C12Q 2600/154; C12Q 2600/118; A61K 31/5377

(57) ABSTRACT

Methods for identifying patients with high risk (progression, invasion, metastasis) squamous cell carcinoma using methylation and/or expression assays are disclosed. Also provided are methods of treating such high risk cancers with ALK inhibitors combined with anti-EGFR based therapies, as the data show a correlation between ALK hypomethylation and EGFR activation.

### PRIORITY CLAIM

This application claims benefit of priority to U.S. Provisional Application Ser. No. 62/511,704, filed May 26, 2017, the entire contents of which are hereby incorporated by reference.

### BACKGROUND

### 1. Field

The present disclosure relates to the fields of molecular biology, oncology and medicine. More particularly, the disclosure relates to the detection and treatment of cancers susceptible to anaplastic lymphoma kinase (ALK)-directed therapies.

### 2. Related Art

Head and neck cancer is the 8^{th} most common cancer in the United States whose incidence is ever-increasing and yet remains one of the most under-studied and under-funded cancer types (Cancer Facts and Figures, 2012; 2015). A total of 48,330 new cases and 9,570 deaths are estimated in 2016 (Cancer Facts and Figures, 2012). Greater than 90% of head and neck cancers are squamous cell carcinoma that arise from the oral cavity (OSCC), oral pharynx (OPSCC), and larynx (Huang et at., 2013), In general, OSCC are primarily associated with tobacco use and alcohol consumption (˜80%), whereas OPSCC is primarily due to infection with human papilloma virus (HPV; HPV-16 and HPV-18) (Huang et al., 2013). While both etiologies are found in both oral and oropharyngeal tumors, HPV related cancer comprise approximately 15% of total cases whereas tobacco and alcohocol related tumors comprise the majority of HNSCC (Cancer Facts and Figures, 2012). The incidence of HNSCC is increasing worldwide. Currently, HNSCC patients are treated with surgery, chemotherapy, radiotherapy or combination. However, these treatment modalities are less effective with advanced disease involving lymph node invasion, which is associated with a five-year survival as low as 34% (Cancer Facts and Figures, 2012). A major factor in decreased survival is resistance to chemotherapy. New lines of therapy are therefore needed to improve the survival rate of HNSCC patients,

In the effort to discover novel molecular factors that contribute to advanced HNSCC and may serve as therapeutic targets, the inventors' recent studies demonstrated that anaplastic lymphoma kinase (ALK) is epigenetically deregulated in late-stage, oral SCC (OSCC) and may play a role in OSCC invasiveness (Huang et al., 2013). They found that invasive OSCC tumors with lymph node metastasis exhibited significantly lower ALK promoter methylation compared to non-invasive OSCC, suggesting that differential ALK promoter methylation affecting expression may predict the development of metastatic OSCC (Huang et al., 2013). Correspondingly, our analysis of head and neck cancer cohort in The Cancer Genome Atlas (TCGA) revealed a significant increase (p<0.01) of ALK expression in tumors compared to normal controls and in an inverse relationship between ALK gene methylation and expression (Huang et at., 2013). These studies suggested that ALK may be a potential therapeutic target for advanced OSCC. Uniquely, the inventors identified full-length ALK as a marker and therapeutic target for HNSCC. Traditionally ALK fusion proteins are the driving factor behind malignancies. Notably, no ALK fusion proteins were identified in the OSCC cohort evaluated by the inventors. This was validated in TCGA that showed an increase in full-length ALK expression and no ALK fusions (Huang et al., 2013). ALK expression is found during development and in tissues of neuronal origin. AIK is not expressed in epithelial-derived tissues. Thus, expression of full length ALK in HNSCC was non-obvious and only identified via a screen of differentially methylated gene promoters in OSCC with nodal disease compared to OSCC with no nodal disease.

ALK fusion proteins were identified as a therapeutic target for non-small cell lung cancer (NSCLC) in 2007 when Soda and colleagues discovered an inversion on chromosome arm 2p resulting in fusion of the 5′ end of echinoderm microtubule-associated protein-like 4 (EML4) to 3₀ALK yielding the EML4-ALK fusion gene (Horn & Pao, 2009; Lindeman et al., 2013). However full length ALK expression due to point mutations and constitutive activation were reported in numerous cancers of mesenchymal origin including neuroblastoma, neuroectodermal tumors, melanoma and glioblastoma (Lindeman et al., 2013; Webb et al., 2009; Powers et al., 2002; Miyake et al., 2002; Shao et al., 2002; Wellstein, 2012; Grzelinski et at., 2009; Wang et al., 2011). NSCLCs expressing EML4-ALK fusion protein respond very well to the ALK small molecule inhibitor, Crizotinib. However, a subgroup eventually develops resistance to ALK inhibition via induction of epidermal growth factor receptor (EGFR) bypass signaling which, like ALK, signals through AKT. RAS, and STATS (Wang et al., 2011; Katayama et al., 2012; 2011; Koivunen et al., 2008)

Approximately 90% of HNSCC express EGFR, yet EGFR inhibitors have yielded little to no efficacy in clinical trials (Moon et al,, 2010; Petrelli et al., 2014). Studies evaluating Erlotinib, a small molecule inhibitor of EGFR, found that a mere 10-15% of oral cancer patients with stage I disease responded to Erlotinib (Tsien et al., 2012). Moreover, patients with stages II, III, and IV disease failed to respond entirely (Tsien el al., 2012). The reason for these unexpected failures of EGFR targeted therapies remains unclear. The inventors demonstrate that full length ALK signaling forms compensatory bypass signaling and that co-targeting ALK and EGFR is necessary to effectively block downstream signaling cascades. Thus OSCC with full length ALK expression may be sensitive to this therapeutic strategy with ALK promoter methylation and expression serving as a biomarker of the subset of susceptible lesions.

### SUMMARY

Thus, in accordance with the present disclosure, there is provided a method of detecting assessing risk of progression, tissue invasion and/or metastasis in a subject having a squamous cell carcinoma comprising assessing, in a cell containing sample from said subject, the methylation status of one or more of the following promoter regions, or the expression of one or more of the following genes:

| | |
|---|---|
| ALK | Anaplastic Lymphoma Kinase |
| HIF3A | Hypoxia Inducible Factor 3 Alpha |
| TRAF7 | TNF Receptor Associated Factor 7 |
| PTK6 | Protein Tyrosine Kinase 6 |
| TNFRSF10C | Tumor Necrosis Factor Receptor Superfamily |
| | Member 10C |
| NCAM1 | Neural Cell Adhesion Molecule 1 |
| CHL1 | Neural Cell Adhesion Molecule L1-like Protein |
| F7 | Coagulation Factor 7 |
| CCDC92 | Coiled-Coil Domain Containing 92 |
| SLC9A3 | Solute Carrier Family 9 Member 3 |
| IRS4 | Insulin Receptor Substrate 4 |

wherein hypomethviation or overexpression, compared to a subject with stable, non-invasive and or non-metastatic squamous cell carcinoma, of one or more said promoters indicates greater than average risk of progression and/or metastasis. In particular, ALK, IRS4, and PTK6 may be assessed for hypomethylation and/or overexpression, optionally further including assessing EGFR overexpression. Two, 3, 4, 5, 6, 7, 8, 9 10 or all 11 of said promoter regions may be assessed. Methylation may be measured by pyrosequecing or whole genome bisulfite conversion/amplification followed by targeted next generation sequencing or pyrosequencing, and/or wherein expression may he measured by FlexMap-based branched DNA probes, microfluidic PCR or droplet digital PCR.

The subject may have oral squamous cell carcinoma or head & neck squamous cell carcinoma. The subject may be a human or a non-human mammal. The cell containing sample may be saliva. Assessing may be repeated a second time, The squamous cell carcinoma may be early stage, late stage, metastatic, recurrent and/or drug resistant. The method may further comprise treating said subject with an ALK inhibitor. The method may also further comprise treating said subject with a second anti-cancer therapy, such as a chemotherapy, a radiotherapy, an immunotherapy, a toxin therapy and/or surgery. The second anti-cancer therapy is an anti-EGFR therapy, such as gefitnib, erlotinib, lapatinib, cetuximab, panitumumab, vandetanib, necitumumab, or osimertinib.

Treating may comprise administration to a tumor site, or local or regional to a tumor site, may comprise systemic administration, may comprise multiple administrations of said anti-EGFR therapy, and/or may comprise multiple administrations of said ALK inhibitor. The patients may be stratified based on salivary DNA methylation/RNA expression signatures for personalized targeted therapy against ALK and in combination with EGFR targeted drugs.

It is contemplated that any method or composition described herein can be implemented with respect to any other method or composition described herein.

Other objects, features and advantages of the present disclosure will become apparent from the following detailed description. It should be understood, however, that the detailed description and the specific examples, while indicating specific embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

The following drawings farm part of the present specification and are included to further demonstrate certain aspects of the present disclosure. The disclosure may be better understood by reference to one or more of these drawings in combination with the detailed description of specific embodiments presented herein.

The patent or application file contains at least one drawing executed in color. Copies of this patent or patent application publication with color drawing(s) will be provided by the Office upon request and payment of the necessary fee.

FIGS. 1A-C. FIG. 1A: Immunohistochemical analysis of activated, phosphorylated ALK phospho-ALK) in human OSCC tumors (4×). High phospho-ALK expression is illustrated in Stage IV OSCCs (panels a & b). Low phospho-ALK expression is illustrated in Stage I OSCCs (panels c & d). Normal tongue tissue (panel c) shows some artifactual staining due to tissue folding (panel e). Relative quantification is shown in panel f. FIG. 1B: Immunofluorescent detection of total and activated (phospho-ALK) in OSCC cell lines (40×). Ca127 cells stained for total and phospho-ALK, panels a & b respectively; HSC3 cells stained for total and phospho-ALK, panels c & d respectively. FIG. 1C: Immunohistochemical analysis of HSC3-derived tumors (20X). Representative H & E staining of HSC3-derived tumors is shown in panel a and corresponding phospho-ALK staining is shown in panel b.

FIGS. 2A-D. FIG. 2A: Cell viability assay of HSC3 cells treated with Gefitinib (500 nM), TAE684 (500 nM), or bath. FIG. 2B: Cell viability assay of Ca127 cells treated with Gefitinib (500 nM), TAE684 (500 nM), or both. FIG. 2C: Assay of HSC3 cells treated with Gefitinib (500 nM), TAE684 (500 nM), or both. FIG. 2D: Wound-healing assay of Ca127 cells treated with Gefitinib (500 nM), TAE684 (500 nM), or both. *p<0.05, **p<0.01, ***p<0.001 treatment compared to control. #p<0.05, ##p<0.01, ###p<0.001 Gefitinib compared to combination treatment. This data represents four replicates and error bars indicate SD.

FIGS. 3A-B. (FIG. 3A) HSC3 mouse xenografts treated daily with Gefitinib (100 mg/kg); TAE684 (10 mg/kg), or both. No change in tumor growth is seen with TAE684 alone as compared to control. Significant reduction in tumor volume compared to control is seen as early as day 6 with Gefitinib alone (57% of control) and day 4 with co-treatment (35% of control). By day 10, a significant reduction in tumor volume between Gefitinib and co-treatments is seen. *p<0.05, **p<0.01, ***p<0.001 treatments compared to control. ##p<0.01, ###P<0.001 Gefitinib compared to co-treatments. This data represents five replicates and error bars indicate SD. (FIG. 3B) Same experiment as FIG. 3A, showing day 14 final tumor volumes.

FIGS. 4A-B. Western blot analysis of EGFR signaling components in OSCC cell lines treated for six hours with Gefitinib (500 nM), TAE684(500 nM), or both. FIG. 4A: HSC3 cells treated with Gefitinib demonstrate a reduced ratio of phosphorylated EGFR (p-EGFR) to total EGFR. An additive reduction of p-EGFR/EGFR is seen with a combination treatment (Combo) of TAE684 and Gefitinib. The ratio of phosphorylated AKT (p-AKT) to total AKT is significantly reduces with individual treatments and abolished with combination treatments. No change in the ratio of phosphorylated ERK1/2 (p-ERK1/2) to total ERK1/2 is seen. Lone treatment with the ALK inhibitor, TAE684, significantly induces activation of STAT3 as reflected in the ratio of phosphorylated STAT3 (p-STAT3) to total STAT3. This induction is reversed with combination treatments of TAE684 and Gefitinib. FIG. 4B: Ca127 cells treated with Gefitinib demonstrate no significant changes in the ratio of p-EGFR to total EGFR. Gefitinib alone abolished p-AKT and TAE684 alone significantly reduced the ratio of p-AKT to total AKT. Similarly, combination treatment of Gefitinib and TAE684 (Combo) abolished p-AKT and no change in the ratio of p-ERK1/2 to total ERK1/2 is seen in Ca127 cells. Although a trend is seen with an increase in the p-STAT3 to total STAT3 ratio is seen with all treatments, no significant changes were detected. *p<0.05, **p<0.01, ***p<0.001 denote significance between treatments.

### DETAILED DESCRIPTION

As discussed above, approximately 90% of HNSCC express EGFR, yet EGFR inhibitors have yielded little to no efficacy in clinical trials (Moon et c.d., 2010; Petrelli et al., 2014). For example, the small molecule inhibitor of EGFR, Erlotinib, exhibited a response rate of only 10-15% in oral cancer patients with stage I disease (Tsien et al., 2012), while later stage patients failed to respond entirely (Tsien et al., 2012). No explanation has been provided for this limited efficacy.

The inventors hypothesize that parallel signaling pathways may compensate for EGFR inhibition, rendering these type of treatments ineffective. in this study, the inventors tested the hypothesis that EGFR inhibition is thwarted by ALK parallel signaling in tumors that progress clinically. They assessed the effects of ALK and EGFR inhibition in ALK expressing OSCC cell lines and in a mouse OSCC xenograft model. They further demonstrated anti-proliferative and signaling effects in vitro and significant tumor growth inhibition in vivo. These and other aspects of the disclosure are reproduced below.

### I. SQUAMOUS CELL CARCINOMA

Squamous cell carcinoma (SCC or SqCC), also known as squamous cell cancer, one of the main types of skin cancer that begins from squamous cells in the skin. Cancers that involve the anus, cervix, head and neck (e.g., oral), and vagina are also most often squamous cell cancers. The esophagus, urinary bladder, prostate, and lung are other possible sites.

Frequent exposure to direct, strong sunlight without adequate sunscreen protection is a risk factor for skin cancer. Despite sharing the name squamous cell carcinoma, the SCCs of different body sites can show differences in their presenting symptoms, natural history, prognosis, and response to treatment.

The tumor grows relatively slowly, but unlike basal-cell carcinoma (BCC), squamous cell carcinoma (SCC) has a substantial risk of metastasis. Risk of metastasis is higher in SCC arising in scars, on the lower lips or mucosa, and occurring in immunosuppressed patients. About one-third of lingual and mucosal tumors metastasize before diagnosis (these are often related to tobacco and alcohol use). Human papillomavirus infection (HPV) has been associated with SCC of the oropharynx, lung, fingers, and anogenital region.

Ninety percent of cases of head and neck cancer (cancer of the mouth, nasal cavity, nasopharynx, throat and associated structures) are of squamous cell origin, i.e., SCC. Squamous cell carcinomas of the head and neck have been found to have a greater risk of metastasis to the local nervous system, lymphatic system, hence possibly reducing treatment efficacy.

Carcinomas of the esophagus were found in one study to have a 58% mean rate of metastasis to local lymph nodes. In the same study, the number of lymph nodes compromised was correlated with a decrease of the survival rate. The study found that in cases of lymphatic metastasis, the mean 5-year survival rate was 49.5%, with a decrease for every lymph node compromised.

### II. DIAGNOSTIC AND THERAPEUTIC METHODS

A. ALK Network Biomarkers

The present inventors have developed a set of prognostic biomarkers that can be used alone or in combinations to identify OSCC that will ultimately invade loco regional tissues and respond to ALK targeted therapies In particular, the inventors have discovered promoter hypomethylation of a network of genes (n=11) associated with anaplastic lymphoma kinase (ALK) signaling in late-stage oral squamous cell carcinoma (OSCC) that metastasize to lymph nodes (stage III and IV). They term this network the “ALK Network,” which drives increased expression of wild-type ALK in OSCC that progress clinically. To date, other tumor types that are known to be ALK driven are a result of ALK fusion proteins or activating mutations. In contrast, the present studies identify altered promoter methylation as facilitation expression and activity of wild-type ALK in OSCC. Most OSCC patients (˜60%) have advanced disease at the time of their diagnosis thereby reducing their five year survival rate to as low as 34%, This is due to late detection and due to a lack of efficacious therapies to treat advanced OSCC. If tumors are detected at an earlier stage (stage I or II) then surgical excision and/or chemotherapy and radiation therapy yield an 80% five-year survival rate. To date, no efficacious therapies exist for advanced tumors that are not fully operable or have metastasized. Salivary diagnostics are a proven method of evaluating gene expression. This technology, looking at promoter methylation analysis and gene/protein expression profiling of the ALK Network in saliva specimens, allows a rapid, non-surgical means of identifying patients with tumors that are invasive and should respond to therapies targeting the ALK Network.

Preclinical studies using ALK Network-guided therapy demonstrated significant efficacy of targeting ALK in OSCC yielding a 65% reduction in xenografted tumor volumes in a mere 14 days of treatment when combined with the EGFR inhibitor, Gefitinib. Notably, co-targeting ALK and EGFR is significantly more efficacious than either tyrosine kinase alone. There are multiple FDA approved drugs that target ALK signaling and in use to treat other cancers that are driven by ALK fusions and mutations. This technology identifies OSCC patients who may respond to commercially available ALK inhibitors.

TABLE A
OSCC Hypomethylation Biomarker Genes

| | |
|---|---|
| ALK | Anaplastic Lymphoma Kinase |
| HIF3A | Hypoxia Inducible Factor 3 Alpha |
| TRAF7 | TNF Receptor Associated Factor 7 |
| PTK6 | Protein Tyrosine Kinase 6 |
| TNFRSF10C | Tumor Necrosis Factor Receptor Superfamily |
| | Member 10C |
| NCAM1 | Neural Cell Adhesion Molecule 1 |
| CHL1 | Neural Cell Adhesion Molecule L1-like Protein |
| F7 | Coagulation Factor 7 |
| CCDC92 | Coiled-Coil Domain Containing 92 |
| SLC9A3 | Solute Carrier Family 9 Member 3 |
| IRS4 | Insulin Receptor Substrate 4 |

There is also disclosed here in an ALK-EGFR Subnetwork that includes ALK, EGFR, IRS4, and PTK6. Hypomethylation is seen only in ALK, IRS4, and PTK6, but this works in concert with over-expressed EGFR.

B. Testing Methods

Tests can be carried out on any suitable sample that yields squamous cells or squamous cell nucleic acids. Particular samples which can be used include tissue specimens, biopsy specimens, surgical specimens, saliva, nasal mucosa, leukoplakia, erythroplakia, leukoerythroplakia and cytological specimens. It may be beneficial to extract nucleic acids from the cells prior to testing. Some techniques of testing may not require pre-extraction, e.g., saliva. Some testing may be done on proteins which may or may not be extracted from the cells prior to testing for particular detection techniques.

1. EXPRESSION

a. Protein Based Assays

The present invention will utilize any of a variety of methods to assess protein expression. Samples containing proteins include fluid samples such as blood, serum, sputum, nipple aspirate and ascites. Also, immunohistochemistry utilizes tissue sections which are specially prepared for analysis using antibody probes. The following provides a general discussion of these embodiments.

i. Immunologic Detection

One such approach is to perform protein identification with the use of antibodies. As used herein, the term “antibody” is intended to refer broadly to any immunologic binding agent such as IgG, IgM, IgA, IgD and IgE. Generally, IgG and/or IgM are preferred because they are the most common antibodies in the physiological situation and because they are most easily made in a laboratory setting. The term “antibody” also refers to any antibody-like molecule that has an antigen binding region, and includes antibody fragments such as Fab′, Fab, F(ab′)₂, single domain antibodies (DABs), Fv, scFv (single chain Fv), and the like. The techniques for preparing and using various antibody-based constructs and fragments are well known in the art. Means for preparing and characterizing antibodies, both polyclonal and monoclonal, are also well known in the art (See, e.g., Antibodies: A. Laboratory Manual, Cold Spring Harbor Laboratory, 1988; incorporated herein by reference). In particular, antibodies to calcyclin, calpactin I light chain, astrocytic phosphoprotein PEA-15 and tubulin-specific chaperone A are contemplated.

In accordance with the present invention, immunodetection methods are provided. Some immunodetection methods include enzyme linked immunosorbent assay (ELISA), radioimmunoassay (RIA), immunoradiometric assay, fluoroimmunoassay, chemilluminescent assay, bioluminescent assay, and Western blot to mention a few. The steps of various useful immunodetection methods have been described in the scientific literature, such as, e.g., Doolittle & Ben-Zeev (1999); Gulbis & Galand (1993); De Jager et al. (1993); and Nakamura et al. (1987), each incorporated herein by reference.

In general, the immunobinding methods include obtaining a sample suspected of containing a relevant polypeptide, and contacting the sample with a first antibody under conditions effective to allow the formation of immunocomplexes. In terms of antigen detection, the biological sample analyzed may be any sample that is suspected of containing an antigen, such as, for example, a tissue section or specimen, a homogenized tissue extract, a cell, or even a biological fluid.

Contacting the chosen biological sample with the antibody under effective conditions and for a period of time sufficient to allow the formation of immune complexes (primary immune complexes) is generally a matter of simply adding the antibody composition to the sample and incubating the mixture for a period of time long enough for the antibodies to form immune complexes with, i.e., to bind to, any antigens present. After this time, the sample-antibody composition, such as a tissue section, ELISA plate, dot blot or western blot, will generally be washed to remove any non-specifically bound antibody species, allowing only those antibodies specifically bound within the primary immune complexes to be detected.

In general, the detection of immunocomplex formation is well known in the art and may be achieved through the application of numerous approaches. These methods are generally based upon the detection of a label or marker, such as any of those radioactive, fluorescent, biological and enzymatic tags, Patents concerning the use of such labels include U.S. Pat. Nos. 3,817,837; 3,850,752; 3,939,350; 3,996,345; 4,277,437; 4,275,149 and 4,366,241, each incorporated herein by reference. Of course, one may find additional advantages through the use of a secondary binding ligand such as a second antibody and/or a biotin/avidin ligand binding arrangement, as is known in the art.

The antibody employed in the detection may itself be linked to a detectable wherein one would then simply detect this label, thereby allowing the amount of the primary immune complexes in the composition to be determined. Alternatively, the first antibody that becomes bound within the primary immune complexes may be detected by means of a second binding ligand that has binding affinity for the antibody. In these cases, the second binding ligand may be linked to a detectable label. The second binding ligand is itself often an antibody, which may thus be termed a “secondary” antibody. The primary immune complexes are contacted with the labeled, secondary binding ligand, or antibody, under effective conditions and for a period of time sufficient to allow the formation of secondary immune complexes. The secondary immune complexes are then generally washed to remove any non-specifically bound labeled secondary antibodies or ligands, and the remaining label in the secondary immune complexes is then detected.

Further methods include the detection of primary immune complexes by a two-step approach. A second binding ligand, such as an antibody, that has binding affinity for the antibody is used to form secondary immune complexes, as described above. After washing, the secondary immune complexes are contacted with a third binding ligand or antibody that has binding affinity for the second antibody, again under effective conditions and for a period of time sufficient to allow the formation of immune complexes (tertiary inunune complexes). The third ligand or antibody is linked to a detectable label, allowing detection of the tertiary immune complexes thus formed, This system may provide for signal amplification if this is desired.

One method of immunodetection designed by Charles Cantor uses two different antibodies. A first step biotinylated, monoclonal or polyclonal antibody is used to detect the target antigen(s), and a second step antibody is then used to detect the biotin attached to the complexed biotin. In that method the sample to be tested is first incubated in a solution containing the first step antibody. If the target antigen is present, some of the antibody binds to the antigen to form a biotinylated antibody/antigen complex.. The antibody/antigen complex is then amplified by incubation in successive solutions of streptavidin (or avidin), biotinylated DNA, and/or complementary biotinylated DNA, with each step adding additional biotin. sites to the antibody/antigen complex. The amplification steps are repeated until a. suitable level of amplification is achieved, at which point the sample is incubated in a solution containing the second step antibody against biotin. This second step antibody is labeled, as for example with an enzyme that can be used to detect the presence of the antibody/antigen complex by histoenzymology using a chromogen substrate. With suitable amplification, a conjugate can be produced which is macroscopically visible.

Another known method of immunodetection takes advantage of the immuno-PCR (Polymerase Chain Reaction) methodology. The PCR method is similar to the Cantor method up to the incubation with biotinylated DNA, however, instead of using multiple rounds of streptavidin and biotinylated DNA incubation, the DNA/biotin/streptavidin/antibody complex is washed out with a low pH or high salt buffer that releases the antibody. The resulting wash solution is then used to carry out a PCR reaction with suitable primers with appropriate controls. At least in theory, the enormous amplification capability and specificity of PCR can be utilized to detect a single antigen molecule.

Immunoassays are in essence binding assays. Certain immunoassays are the various types of enzyme linked immunosorbent assays (ELISAs) and radioimmunoassays (RIA) known in the art. However, it will be readily appreciated that detection is not limited to such techniques, and Western blotting, dot blotting, FACS analyses, immunohistochemistry and the like may also be used.

The antibodies of the present invention may be used in conjunction with both fresh-frozen and/or formalin-fixed, paraffin-embedded tissue blocks prepared for study by immunohistochemistry (IHC). The method of preparing tissue blocks from these particulate specimens has been successfully used in previous IHC studies of various prognostic factors, and/or is well known to those of skill in the art (Brown et al., 1990; Abbondanzo et al., 1999; Allred et al., 1990).

Also contemplated in the present invention is the use of immunohistochemistry. This approach uses antibodies to detect and quantify antigens in intact tissue samples. Generally, frozen-sections are prepared by rehydrating frozen “pulverized” tissue at room temperature in phosphate buffered saline (PBS) in small plastic capsules; pelleting the particles by centrifugation; resuspending them in a viscous embedding medium (OCT); inverting the capsule and pelleting again by centrifugation; snap-freezing in −70° C. isopentane; cutting the plastic capsule and removing the frozen cylinder of tissue; securing the tissue cylinder on a cryostat microtome chuck; and cutting 25-50 serial sections.

Permanent-sections may be prepared by a similar method involving rehydration of the 50 mg sample in a plastic microfuge tube; pelleting; resuspending in 10% fonnalin for 4 hours fixation; washing/pelleting; resuspending in warm 2.5% agar; pelleting; cooling in ice water to harden the agar; removing the tissue/agar block from the tube; infiltrating and/or embedding the block in paraffin; and cutting up to 50 serial permanent sections.

In an exemplary ELISA, the antibodies of the invention are immobilized onto a selected surface exhibiting protein affinity, such as a well in a polystyrene microtiter plate. Then, a test composition suspected of containing the antigen, such as a clinical sample, is added to the wells. After binding and washing to remove non-specifically bound immune complexes, the bound antigen may be detected. Detection is generally achieved by the addition of another antibody that is linked to a detectable label. This type of ELISA is a simple “sandwich ELISA.” Detection may also be achieved by the addition of a second antibody, followed by the addition of a third antibody that has binding affinity for the second antibody, with the third antibody being linked to a detectable label.

In another exemplary ELISA, the samples suspected of containing the antigen are immobilized onto the well surface and then contacted with the anti-ORF message and anti-ORF translated product antibodies of the invention. After binding and washing to remove non-specifically bound immune complexes, the bound anti-ORF message and anti-ORF translated product antibodies are detected. Where the initial anti-ORF message and anti-ORF translated product antibodies are linked to a detectable label, the immune complexes may be detected directly. Again, the immune complexes may be detected using a second antibody that has binding affinity for the first anti-ORF message and anti-ORF translated product antibody, with the second antibody being linked to a detectable label.

Another ELISA in which the antigens are immobilized involves the use of antibody competition in the detection. In this ELISA, labeled antibodies against an antigen are added to the wells, allowed to bind, and detected by means of their label. The amount of an antigen in an unknown sample is then determined by mixing the sample with the labeled antibodies against the antigen during incubation with coated wells. The presence of an antigen in the sample acts to reduce the amount of antibody against the antigen available for binding to the well and thus reduces the ultimate signal. This is also appropriate for detecting antibodies against an antigen in an unknown sample, where the unlabeled antibodies bind to the antigen-coated wells and also reduces the amount of antigen available to bind the labeled antibodies.

“Under conditions effective to allow immune complex (antigen/antibody) formation” means that the conditions preferably include diluting the antigens and/or antibodies with solutions such as BSA, bovine gamma globulin (BGG) or phosphate buffered saline (PBS)/Tween. These added agents also tend to assist in the reduction of nonspecific background. The “suitable” conditions also mean that the incubation is at a temperature or for a period of time sufficient to allow effective binding. Incubation steps are typically from about 1 to 2 to 4 hours or so, at temperatures preferably on the order of 25° C. to 27° C., or may be overnight at about 4° C. or so.

ii. Mass Spectrometry

By exploiting the intrinsic properties of mass and charge, mass spectrometry (MS) can resolved and confidently identified a wide variety of complex compounds, including proteins. Traditional quantitative MS has used electrospray ionization (ESI) followed by tandem MS (MS/MS) (Chen et al., 2001; Zhong et al., 2001; Wu et al., 2000) while newer quantitative methods are being developed using matrix assisted laser desorption/ionization (MALI) followed by time of flight (TOF) MS (Bucknall et al., 2002; Mirgorodskaya et al., 2000; Gobom et al., 2000). In accordance with the present invention, one can generate mass spectrometry profiles that are useful for grading gliomas and predicting glioma patient survival, without regard for the identity of specific proteins. Alternatively, given the established links with calcyclin, calpactin I light chain, astrocytic phosphoprotein PEA-15 and tubulin-specific chaperone A, mass spectrometry may be used to look for the levels of these proteins particularly.

ESL ESI is a convenient ionization technique developed by Fenn and colleagues (Fenn et al., 1989) that is used to produce gaseous ions from highly polar, mostly nonvolatile biomolecules, including lipids. The sample is injected as a liquid at low flow rates (1-10 μL/min) through a capillary tube to which a strong electric field is applied. The field generates additional charges to the liquid at the end of the capillary and produces a fine spray of highly charged droplets that are electrostatically attracted to the mass spectrometer inlet. The evaporation of the solvent from the surface of a droplet as it travels through the desolvation chamber increases its charge density substantially. When this increase exceeds the Rayleigh stability limit, ions are ejected and ready for MS analysis.

A typical conventional ESI source consists of a metal capillary of typically 0.1-0.3 mm in diameter, with a tip held approximately 0.5 to 5 cm (but more usually 1 to 3 cm) away from an electrically grounded circular interface having at its center the sampling orifice, such as described by Kabarle et al. (1993). A potential difference of between 1 to 5 kV (but more typically 2 to 3 kV) is applied to the capillary by power supply to generate a high electrostatic field (10⁶ to 10⁷ V/m) at the capillary tip. A sample liquid carrying the analyte to be analyzed by the mass spectrometer is delivered to tip through an internal passage from a suitable source (such as from a chromatograph or directly from a sample solution via a liquid flow controller). By applying pressure to the sample in the capillary, the liquid leaves the capillary tip as small, highly electrically-charged droplets, and further undergoes desolvation and breakdown to form single or multicharged gas phase ions in the form of an ion beam. The ions are then collected by the grounded (or negatively charged) interface plate and led through an orifice into an analyzer of the mass spectrometer. During this operation, the voltage applied to the capillary is held constant. Aspects of construction of ESI sources are described, for example, in U.S. Pat. Nos. 5,838,002; 5,788,166; 5,757,994; RE 35,413; and 5,986,258.

ESI/MS/MS. In ESI tandem mass spectroscopy (ESI/MS/MS), one is able to simultaneously analyze both precursor ions and product ions, thereby monitoring a single precursor product reaction and producing (through selective reaction monitoring (SRM)) a signal only when the desired precursor ion is present. When the internal standard is a stable isotope-labeled version of the analyte, this is known as quantification by the stable isotope dilution method. This approach has been used to accurately measure pharmaceuticals (Zweigenbaum et al., 2000; Zweigenbaum et al., 1999) and bioactive peptides (Desiderio et al., 1996; Lovelace et al., 1991). Newer methods are performed on widely available MALDI-TOF instruments, which can resolve a wider mass range and have been used to quantify metabolites, peptides, and proteins. Larger molecules such as peptides can be quantified using unlabeled homologous peptides as long as their chemistry is similar to the analyte peptide (Duncan et all., 1993; Bucknall et al., 2002). Protein quantification has been achieved by quantifying tryptic peptides (Mirgorodskaya et al., 2000). Complex mixtures such as crude extracts can be analyzed, but in some instances sample clean-up is required (Nelson et al., 1994; Gobom et al., 2000).

SIMS. Secondary ion mass spectroscopy, or SIMS, is an analytical method that uses ionized particles emitted from a surface for mass spectroscopy at a sensitivity of detection of a few parts per billion. The sample surface is bombarded by primary energetic particles, such as electrons, ions (e.g., O, Cs), neutrals or even photons, forcing atomic and molecular particles to be ejected from the surface, a process called sputtering. Since some of these sputtered particles carry a charge, a mass spectrometer can be used to measure their mass and charge. Continued sputtering permits measuring of the exposed elements as material is removed. This in turn permits one to construct elemental depth profiles. Although the majority of secondary ionized particles are electrons, it is the secondary ions which are detected and analysis by the mass spectrometer in this method.

LD-MS and LDLPMS. Laser desorption mass spectroscopy (LD-MS) involves the use of a pulsed laser, which induces desorption of sample material from a sample site—effectively, this means vaporization of sample off of the sample substrate. This method is usually only used in conjunction with a mass spectrometer, and can be performed simultaneously with ionization if one uses the right laser radiation wavelength.

When coupled with Time-of-Flight (TDF) measurement, LD-MS is referred to as LDLPMS (Laser Desorption Laser Photoionization Mass Spectroscopy). The LDLPMS method of analysis gives instantaneous volatilization of the sample, and this form of sample fragmentation permits rapid analysis without any wet extraction chemistry. The LDLPMS instrumentation provides a profile of the species present while the retention time is low and the sample size is small. In LDLPMS, an impactor strip is loaded into a vacuum chamber. The pulsed laser is fired upon a certain spot of the sample site, and species present are desorhed and ionized by the laser radiation. This ionization also causes the molecules to break up into smaller fragment-ions. The positive or negative ions made are then accelerated into the flight tube, being detected at the end by a microchannel plate detector. Signal intensity, or peak height, is measured as a function of travel time. The applied voltage and charge of the particular ion determines the kinetic energy, and separation of fragments is due to different size causing different velocity. Each ion mass will thus have a different flight-time to the detector.

One can either form positive ions or negative ions for analysis. Positive ions are made from regular direct photoionization, but negative ion formation requires a higher powered laser and a secondary process to gain electrons. Most of the molecules that come off the sample site are neutrals, and thus can attract electrons based on their electron affinity. The negative ion formation process is less efficient than forming just positive ions. The sample constituents will also affect the outlook of a negative ion spectra.

Other advantages with the LDLPMS method include the possibility of constructing the system to give a quiet baseline of the spectra because one can prevent coevolved neutrals from entering the flight tube by operating the instrument in a linear mode. Also, in environmental analysis, the salts in the air and as deposits will not interfere with the laser desorption and ionization. This instrumentation also is very sensitive, known to detect trace levels in natural samples without any prior extraction preparations.

MALDI-TOF-MS. Since its inception and commercial availability, the versatility of MALDI-TOF-MS has been demonstrated convincingly by its extensive use for qualitative analysis. For example, MALDI-TOF-MS has been employed for the characterization of synthetic polymers (Marie et al., 2000; Wu et al., 1998). peptide and protein analysis (Roepstorff et al., 2000; Nguyen et al., 1995), DNA and oligonucleotide sequencing (Miketova et al., 1997; Faulstich et al., 1997; Bentzley et al., 1996), and the characterization of recombinant proteins (Kanazawa et al., 1999; Villanueva et al., 1999). Recently, applications of MALDI-TOF-MS have been extended to include the direct analysis of biological tissues and single cell organisms with the aim of characterizing endogenous peptide and protein constituents (Li et al., 2000; Lynn et al., 1999; Stoeckli et al., 2001; Caprioli et al., 1997; Chaurand et al., 1999; Jespersen et al., 1999).

The properties that make MALDI-TOF-MS a popular qualitative tool its ability to analyze molecules across an extensive mass range, high sensitivity, minimal sample preparation and rapid analysis times—also make it a potentially useful quantitative tool. MALDI-TOF-MS also enables non-volatile and thermally labile molecules to be analyzed with relative ease. It is therefore prudent to explore the potential of MALDI-TOF-MS for quantitative analysis in clinical settings, for toxicological screenings, as well as for environmental analysis. In addition, the application of MALDI-TOF-MS to the quantification of peptides and proteins is particularly relevant. The ability to quantify intact proteins in biological tissue and fluids presents a particular challenge in the expanding area of proteomics and investigators urgently require methods to accurately measure the absolute quantity of proteins. While there have been reports of quantitative MALDI-TOF-MS applications, there are many problems inherent to the MALDI ionization process that have restricted its widespread use (Kazmaier et al., 1998; Horak et al., 2001; Gobom et al., 2000; Desiderio et al., 2000). These limitations primarily stem from factors such as the sample/matrix heterogeneity, which are believed to contribute to the large variability in observed signal intensities for analytes, the limited dynamic range due to detector saturation, and difficulties associated with coupling MALDI-TOF-MS to on-line separation techniques such as liquid chromatography. Combined, these factors are thought to compromise the accuracy, precision, and utility with which quantitative determinations can be made.

Because of these difficulties, practical examples of quantitative applications of MALDI-TOF-MS have been limited. Most of the studies to date have focused on the quantification of low mass analytes, in particular, alkaloids or active ingredients in agricultural or food products (Wang et al., 1999; Jiang et al., 2000; Yang et al., 2000; Wittma.nn et al., 2001), whereas other studies have demonstrated the potential of MALDI-TOF-MS for the quantification of biologically relevant analytes such as neuropeptides, proteins, antibiotics, or various metabolites in biological tissue or fluid (Muddiman et al., 1996; Nelson et al., 1994; Duncan et al., 1993; Gobom et al., 2000; Wu et al., 1997; Mirgorodskaya et al., 2000). In earlier work it was shown that linear calibration curves could be generated by MALDI-TOF-MS provided that an appropriate internal standard was employed (Duncan et al., 1993). This standard can “correct” for both sample-to-sample and shot-to-shot variability. Stable isotope labeled internal standards (isotopomers) give the best result.

With the marked improvement in resolution available on modern commercial instruments, primarily because of delayed extraction (Bahr et al., 1997; Takach et al., 1997), the opportunity to extend quantitative work to other examples is now possible; not only of low mass analytes, but also biopolymers. Of particular interest is the prospect of absolute multi-component quantification in biological samples (e.g., proteomics applications).

The properties of the matrix material used in the MALIN method are critical. Only a select group of compounds is useful for the selective desorption of proteins and polypeptides. A review of all the matrix materials available for peptides and proteins shows that there are certain characteristics the compounds must share to be analytically useful. Despite its importance, very little is known about what makes a matrix material “successful” for MALDI. The few materials that do work well are used heavily by all MALDI practitioners and new molecules are constantly being evaluated as potential matrix candidates. With a few exceptions, most of the matrix materials used are solid organic acids. Liquid matrices have also been investigated, but are not used routinely.

2. Nucleic Acid Based Assays

In another aspect, the invention may further utilize detection of mRNA's to determine gene expression. While labile, mRNAs have the advantage of amplification using techniques such as RT-PCR, allowing very small amounts of target to be accurately quantitated. They will also provide complementary information relating to levels that are separate from the issue of protein degradation. The following is a general discussion of such methods.

i. Hybridization

The use of a probe or primer of between 13 and 100 nucleotides, preferably between 17 and 100 nucleotides in length, or in some aspects of the invention up to 1-2 kilobases or more in length, allows the formation of a duplex molecule that is both stable and selective. Molecules having complementary sequences over contiguous stretches greater than 20 bases in length are generally preferred, to increase stability and/or selectivity of the hybrid molecules obtained. One will generally prefer to design nucleic acid molecules for hybridization having one or more complementary sequences of 20 to 30 nucleotides, or even longer where desired. Such fragments may be readily prepared, for example, by directly synthesizing the fragment by chemical means or by introducing selected sequences into recombinant vectors for recombinant production.

Accordingly, the nucleotide sequences of the invention may be used for their ability to selectively form duplex molecules with complementary stretches of DNAs and/or RNAs or to provide primers thr amplification of DNA or RNA from samples. Depending on the application envisioned, one would desire to employ varying conditions of hybridization to achieve varying degrees of selectivity of the probe or primers for the target sequence.

For applications requiring high selectivity, one will typically desire to employ relatively high stringency conditions to form the hybrids. For example, relatively low salt and/or high temperature conditions, such as provided by about 0.02 M to about 0.10 M NaCl at temperatures of about 50° C. to about 70° C. Such high stringency conditions tolerate little, if any, mismatch between the probe or primers and the template or target strand and would be particularly suitable for isolating specific genes or for detecting specific mRNA transcripts. It is generally appreciated that conditions can be rendered more stringent by the addition of increasing amounts of formamide.

For certain applications it is appreciated that lower stringency conditions are preferred. Under these conditions, hybridization may occur even though the sequences of the hybridizing strands are not perfectly complementary, but are mismatched at one or more positions. Conditions may be rendered less stringent by increasing salt concentration and/or decreasing temperature. For example, a medium stringency condition could be provided by about 0.1 to 0.25 M NaCl at temperatures of about 37° C. to about 55° C., while a low stringency condition could be provided by about 0.15 M to about 0.9 M salt, at temperatures ranging from about 20° C. to about 55° C. Hybridization conditions can be readily manipulated depending on the desired results.

In other embodiments, hybridization may be achieved under conditions of, for example, 50 mM Tris-HCl (pH 8.3), 75 mM KCl, 3 mM MgCl₂. 1.0 mM dithiothreitol, at temperatures between approximately 20° C. to about 37° C. Other hybridization conditions utilized could include approximately 10 mM Tris-HCl (pH 8.3), 50 mM KCl, 1.5 mM MgCl₂, at temperatures ranging from approximately 40° C. to about 72° C.

In certain embodiments, it will be advantageous to employ nucleic acids of defined sequences of the present invention in combination with an appropriate means, such as a label, for determining hybridization. A wide variety of appropriate indicator means are known in the art, including fluorescent, radioactive, enzymatic or other ligands, such as avidin/biotin, which are capable of being detected. in particular embodiments, one may desire to employ a fluorescent label or an enzyme tag such as urease, alkaline phosphatase or peroxidase, instead of radioactive or other environmentally undesirable reagents. In the case of enzyme tags. colorimetric indicator substrates are known that can be employed to provide a detection means that is visibly or spectrophotometrically detectable, to identify specific hybridization with complementary nucleic acid containing samples.

In general, it is envisioned that the probes or primers described herein will be useful as reagents in solution hybridization, as in PCR™, for detection of expression of corresponding genes, as well as in embodiments employing a solid phase. In embodiments involving a solid phase, the test DNA (or RNA) is adsorbed or otherwise affixed to a selected matrix or surface. This fixed, single-stranded nucleic acid is then subjected to hybridization with selected probes under desired conditions. The conditions selected will depend on the particular circumstances (depending, for example, on the G+C content, type of target nucleic acid, source of nucleic acid, size of hybridization probe, etc.). Optimization of hybridization conditions for the particular application of interest is well known to those of skill in the art. After washing of the hybridized molecules to remove non-specifically bound probe molecules, hybridization is detected, and/or quantified, by determining the amount of bound label. Representative solid phase hybridization methods are disclosed in U.S. Pat. Nos. 5,843,663, 5,900,481 and 5,919,626. Other methods of hybridization that may be used in the practice of the present invention are disclosed in U.S. Pat. Nos. 5,849,481, 5,849,486 and 5,851,772 and U.S. Patent Publication 2008/0009439. The relevant portions of these and other references identified in this section of the Specification arc incorporated herein by reference.

ii. In situ Hybridization

In situ hybridization (ISH) is a type of hybridization that uses a labeled complementary DNA or RNA strand (i.e., probe) to localize a specific DNA or RNA sequence in a portion or section of tissue (in situ), or, if the tissue is small enough (e.g. plant seeds, Drosophila embryos), in the entire tissue (whole mount ISH). This is distinct from immunohistochemistry, which localizes proteins in tissue sections. Fluorescent DNA ISH (FISH) can, fhr example, be used in medical diagnostics to assess chromosomal integrity. RNA ISH (hybridization histochemistry) is used to measure and localize mRNAs and other transcripts within tissue sections or whole mounts.

For hybridization histochemistry, sample cells and tissues are usually treated to fix the target transcripts in place and to increase access of the probe. As noted above, the probe is either a labeled complementary DNA or, now most commonly, a complementary RNA (riboprobe). The probe hybridizes to the target sequence at elevated temperature, and then the excess probe is washed away (after prior hydrolysis using RNase in the case of unhybridized, excess RNA probe). Solution parameters such as temperature, salt and/or detergent concentration can be manipulated to remove any non-identical interactions (i.e., only exact sequence matches will remain bound). Then, the probe that was labeled with either radio-, fluorescent- or antigen-labeled bases (e.g., digoxigenin) is localized and quantitated in the tissue using either autoradiography, fluorescence microscopy or immunohistochemistry, respectively. ISH can also use two or more probes, labeled with radioactivity or the other non-radioactive labels, to simultaneously detect two or more transcripts.

iii. Amplification of Nucleic Acids

Nucleic acids used as a template for amplification may be isolated from cells, tissues or other samples according to standard methodologies (Sambrook et al., 2001). In certain embodiments, analysis is perfortned on whole cell or tissue homogenates or biological fluid samples without substantial purification of the template nucleic acid. The nucleic acid may be genomic DNA or fractionated or whole cell RNA. Where RNA is used, it may be desired to first convert the RNA to a complementary DNA.

The term “primer,” as used herein, is meant to encompass any nucleic acid that is capable of priming the synthesis of a nascent nucleic acid in a template-dependent process. Typically, primers are oligonucleotides from ten to twenty and/or thirty base pairs in length, but longer sequences can be employed. Primers may be provided in double-stranded and/or single-stranded form, although the single-stranded form is preferred.

Pairs of primers designed to selectively hybridize to nucleic acids corresponding to any sequence corresponding to a nucleic acid sequence are contacted with the template nucleic acid under conditions that permit selective hybridization. Depending upon the desired application, high stringency hybridization conditions may be selected that will only allow hybridization to sequences that are completely complementary to the primers. In other embodiments, hybridization may occur under reduced stringency to allow for amplification of nucleic acids containing one or more mismatches with the primer sequences. Once hybridized, the template-primer complex is contacted with one or more enzymes that facilitate template-dependent nucleic acid synthesis. Multiple rounds of amplification, also referred to as “cycles,” are conducted until a sufficient amount of amplification product is produced.

The amplification product may be detected or quantified. In certain applications, the detection may be performed by visual means. Alternatively, the detection may involve indirect identification of the product via chemiluminescence, radioactive scintigraphy of incorporated radiolabel or fluorescent label or even via a system using electrical and/or thermal impulse signals (Bellus, 1994).

A number of template dependent processes are available to amplify the oligonucleotide sequences present in a given template sample. One of the best known amplification methods is the polymerase chain reaction (referred to as PCRTM) which is described in detail in U.S. Pat. Nos. 4,683,195, 4,683,202 and 4,800,159, and in Innis et al. (1988), each of which is incorporated herein by reference in their entirety.

A reverse transcriptase PCR™ amplification procedure may be performed to quantify the amount of mRNA amplified. Methods of reverse transcribing RNA into cDNA are well known (see Sambrook et al., 2001). Alternative methods for reverse transcription utilize thermostable DNA polymerases. These methods are described in WO 90/07641. Polymerase chain reaction methodologies are well known in the art. Representative methods of RT-PCR are described in U.S. Pat. No. 5,882,864.

Reverse transcription (RT) of RNA to cDNA followed by quantitative PCR (RT-PCR) can be used to determine the relative concentrations of specific mRNA species isolated from a cell. By determining that the concentration of a specific mRNA species varies, it is shown that the gene encoding the specific mRNA species is differentially expressed. If a graph is plotted in which the cycle number is on the X axis and the log of the concentration of the amplified target DNA is on the Y axis, a curved line of characteristic shape is formed by connecting the plotted points. Beginning with the first cycle, the slope of the line is positive and constant. This is said to be the linear portion of the curve. After a reagent becomes limiting, the slope of the line begins to decrease and eventually becomes zero. At this point the concentration of the amplified target DNA becomes asymptotic to some fixed value. This is said to he the plateau portion of the curve.

The concentration of the target DNA in the linear portion of the PCR amplification is directly proportional to the starting concentration of the target before the reaction began. By determining the concentration of the amplified products of the target DNA in PCR reactions that have completed the same number of cycles and are in their linear ranges, it is possible to determine the relative concentrations of the specific target sequence in the original DNA mixture. If the DNA mixtures are cDNAs synthesized from RNAs isolated from different tissues or cells, the relative abundances of the specific mRNA from which the target sequence was derived can he determined for the respective tissues or cells. This direct proportionality between the concentration of the PCR products and the relative mRNA abundances is only true in the linear range of the PCR reaction.

The final concentration of the target DNA in the plateau portion of the curve is determined by the availability of reagents in the reaction mix and is independent of the original concentration of target DNA. Therefore, the first condition that must be met before the relative abundances of an mRNA species can be determined by RT-PCR for a collection of RNA populations is that the concentrations of the amplified PCR products must be sampled when the PCR reactions are in the linear portion of their curves.

A second condition for an RT-PCR experiment is to determine the relative abundances of a particular mRNA species. Typically, relative concentrations of the amplifiable cDNAs are normalized to some independent standard. The goal of an RT-PCR experiment is to determine the abundance of a particular mRNA species relative to the average abundance of all mRNA species in the sample.

Most protocols for competitive PCR utilize internal PCR standards that are approximately as abundant as the target. These strategies are effective if the products of the PCR amplifications are sampled during their linear phases. If the products are sampled when the reactions are approaching the plateau phase, then the less abundant product becomes relatively over represented, Comparisons of relative abundances made for many different RNA samples, such as is the case when examining RNA samples for differential expression, become distorted in such a way as to make differences in relative abundances of RNAs appear less than they actually are. This is not a significant problem if the internal standard is much more abundant than the target. If the internal standard is more abundant than the target, then direct linear comparisons can be made between RNA samples.

RT-PCR can be performed as a relative quantitative RT-PCR with an internal standard in which the internal standard is an amplifiable cDNA fragment that is larger than the target cDNA fragment and in which the abundance of the mRNA encoding the internal standard is roughly 5-100-fold higher than the mRNA encoding the target. This assay measures relative abundance, not absolute abundance of the respective mRNA species.

Another method for amplification is ligase chain reaction (“LCR”), disclosed in European Application No. 320 308, incorporated herein by reference in its entirety. U.S. Pat. No. 4,883,750 describes a method similar to LCR for binding probe pairs to a target sequence. A method based on PCR™ and oligonucleotide ligase assay (OLA), disclosed in U.S. Pat. No. 5,912,148, may also be used.

Alternative methods for amplification of target nucleic acid sequences that may be used in the practice of the present invention are disclosed in U.S. Pat. Nos. 5,843,650, 5,846,709, 5,846,783, 5,849,546, 5,849,497, 5,849,547, 5,858,652, 5,866,366, 5,916,776, 5,922,574, 5,928,905, 5,928,906, 5,932,451, 5,935,825, 5,939,291 and 5,942,391, GB Application No. 2 202 328, and in PCT Application No. PCI/US89/01025, each of which are incorporated herein by reference in its entirety.

Qbeta Replicase, described in PCT Application No. PCT/US87/00880, may also be used as an amplification method in the present invention. In this method, a replicative sequence of RNA that has a region complementary to that of a target is added to a sample in the presence of an RNA polymerase. The polymerase will copy the replicative sequence which may then be detected.

An isothermal amplification method, in which restriction endonucleases and ligases are used to achieve the amplification of target molecules that contain nucleotide 5′-[alpha-thio]-triphosphates in one strand of a restriction site may also be useful in the amplification of nucleic acids in the present invention (Walker et al., 1992). Strand Displacement Amplification (SDA), disclosed in U.S. Pat. No. 5,916,779, is another method of carrying out isothermal amplification of nucleic acids which involves multiple rounds of strand displacement and synthesis, i.e., nick translation.

Other nucleic acid amplification procedures include transcription-based amplification systems (TAS), including nucleic acid sequence based amplification (NASBA) and 3SR (Kwoh et al., 1989; PCT Application WO 88/10315, incorporated herein by reference in their entirety). European Application No. 329 822 disclose a nucleic acid amplification process involving cyclically synthesizing single-stranded RNA (“ssRNA”), ssDNA, and double-stranded DNA (dsDNA), which may be used in accordance with the present invention.

PCT Application WO 89/06700 (incorporated herein by reference in its entirety) disclose a nucleic acid sequence amplification scheme based on the hybridization of a promoter region/primer sequence to a target single-stranded DNA (“ssDNA”) followed by transcription of many RNA copies of the sequence. This scheme is not cyclic, i.e., new templates are not produced from the resultant RNA transcripts. Other amplification methods include “RACE” and “one-sided PCR” (Frohman, 1990; Ohara et al., 1989).

Following any amplification, it may be desirable to separate the amplification product from the template and/or the excess primer. In one embodiment, amplification products are separated by agarose, agarose-acrylamide or polyacrylamide gel electrophoresis using standard methods (Sambrook et al., 2001). Separated amplification products may be cut out and eluted from the gel for further manipulation. Using low melting point agarose gels, the separated band may be removed by heating the gel, followed by extraction of the nucleic acid.

Separation of nucleic acids may also be effected by chromatographic techniques known in art. There are many kinds of chromatography which may be used in the practice of the present invention, including adsorption, partition, ion-exchange, hydroxylapatite, molecular sieve, reverse-phase, column, paper, thin-layer, and gas chromatography as well as HPLC.

In certain embodiments, the amplification products are visualized. A typical visualization method involves staining of a gel with ethidium bromide and visualization of bands under UV light. Alternatively, if the amplification products are integrally labeled with radio- or fluorometrically-labeled nucleotides, the separated amplification products can be exposed to x-ray film or visualized under the appropriate excitatory spectra.

In one embodiment, following separation of amplification products, a labeled nucleic acid probe is brought into contact with the amplified marker sequence. The probe preferably is conjugated to a chromophore but may be radiolabeled. In another embodiment, the probe is conjugated to a binding partner, such as an antibody or biotin, or another binding partner carrying a detectable moiety.

In particular embodiments, detection is by Southern blotting and hybridization with a labeled probe. The techniques involved in Southern blotting are well known to those of skill in the art (see Sambrook et al., 2001). One example of the foregoing is described in U.S. Pat. No. 5,279,721, incorporated by reference herein, which discloses an apparatus and method for the automated electrophoresis and transfer of nucleic acids. The apparatus permits electrophoresis and blotting without external manipulation of the gel and is ideally suited to carrying out methods according to the present invention.

Various nucleic acid detection methods known in the art are disclosed in U.S. Pat. Nos. 5,840,873, 5,843,640, 5,843,651, 5,846,708, 5,846,717, 5,846,726, 5,846,729, 5,849,487, 5,853,990, 5,853,992, 5,853,993, 5,856,092, 5,861,244, 5,863,732, 5,863,753, 5,866,331, 5,905,024, 5,910,407, 5,912,124, 5,912,145, 5,919,630, 5,925,517, 5,928,862, 5,928,869, 5,929,227, 5,932,413 and 5,935,791, each of which is incorporated herein by reference.

iv. Chip Technologies and Arrays

Specifically contemplated by the present inventors are chip-based DNA technologies such as those described by Hacia et al. (1996) and Shoemaker et al. (1996). Briefly, these techniques involve quantitative methods for analyzing large numbers of genes rapidly and accurately. By tagging genes with oligonucleotides or using fixed probe arrays, one can employ chip technology to segregate target molecules as high density arrays and screen these molecules on the basis of hybridization (see also, Pease et al., 1994; and Fodor et al., 1991). It is contemplated that this technology may be used in conjunction with evaluating the expression level of an mRNA with respect to diagnostic, as well as preventative and treatment methods of the invention.

The present invention may involve the use of arrays or data generated from an array. Data may be readily available. Moreover, an array may be prepared in order to generate data that may then be used in correlation studies.

An array generally refers to ordered macroarrays or microarrays of nucleic acid molecules (probes) that are fully or nearly complementary or identical to a plurality of mRNA molecules or cDNA molecules and that are positioned on a support material in a spatially separated organization. Macroarrays are typically sheets of nitrocellulose or nylon upon which probes have been spatted. Microarrays position the nucleic acid probes more densely such that up to 10,000 nucleic acid molecules can be fit into a region typically 1 to 4 square centimeters. Microarrays can be fabricated by spotting nucleic acid molecules, e.g., genes, oligonucleotides, etc., onto substrates or fabricating oligonucleotide sequences in situ on a substrate. Spotted or fabricated nucleic acid molecules can be applied in a high density matrix pattern of up to about 30 non-identical nucleic acid molecules per square centimeter or higher, e.g. up to about 100 or even 1000 per square centimeter. Microarrays typically use coated glass as the solid support, in contrast to the nitrocellulose-based material of filter arrays. By having an ordered array of complementing nucleic acid samples, the position of each sample can be tracked and linked to the original sample. A variety of different array devices in which a plurality of distinct nucleic acid probes are stably associated with the surface of a solid support are known to those of skill in the art. Useful substrates for arrays include nylon, glass and silicon Such arrays may vary in a number of different ways, including average probe length, sequence or types of probes, nature of bond between the probe and the array surface, e.g. covalent or non-covalent, and the like. The labeling and screening methods of the present invention and the arrays are not limited in its utility with respect to any parameter except that the probes detect expression levels; consequently, methods and compositions may be used with a variety of different types of genes.

Representative methods and apparatus for preparing a microarray have been described, for example, in U.S. Pat. Nos. 5,143,854; 5,202,231; 5,242,974; 5,288,644; 5,324,633; 5,384,261; 5,405,783; 5,412,087; 5,424,186; 5,429,807; 5,432,049; 5,436,327; 5,445,934; 5,468,613; 5,470,710; 5,472,672; 5,492,806; 5,525,464; 5,503,980; 5,510,270; 5,525,464; 5,527,681; 5,529,756; 5,532,128; 5,545,531; 5,547,839; 5,554,501; 5,556,752; 5,561,071; 5,571,639; 5,580,726; 5,580,732; 5,593,839; 5,599,695; 5,599,672; 5,610;287; 5,624,711; 5,631,134; 5,639,603; 5,654,413; 5,658,734; 5.661.028; 5,665,547; 5,667,972; 5,695,940; 5,700,637; 5,744,305; 5,800,992; 5,807,522; 5,830,645; 5,837,196; 5,871,928; 5,847,219; 5,876,932; 5,919,626; 6,004,755; 6,087,102; 6,368,799; 6,383,749; 6,617,112; 6,638,717; 6,720,138, as well as WO 93/17126; WO 95/11995; WO 95/21265; WO 95/21944; WO 95/35505; WO 96/31622; WO 97/10365; WO 97/27317; WO 99/35505; WO 09923256; WO 09936760; W00138580; WO 0168255; WO 03020898; WO 03040410; WO 03053586; WO 03087297; WO 03091426; W003100012; WO 04020085; WO 04027093; EP 373 203; EP 785 280; EP 799 897 and UK 8 803 000; the disclosures of which are all herein incorporated by reference.

It contemplated that the arrays can be high density arrays, such that they contain 100 or more different probes. It is contemplated that they may contain 1000, 16,000, 65,000, 250,000 or 1,000,000 or more different probes. The probes can be directed to targets in one or more different organisms. The oligonucleotide probes range from 5 to 50, 5 to 45, 10 to 40, or 15 to 40 nucleotides in length in some embodiments. In certain embodiments, the oligonucleotide probes are 20 to 25 nucleotides in length.

The location and sequence of each different probe sequence in the array are generally known. Moreover, the large number of different probes can occupy a relatively small area providing a high density array having a probe density of generally greater than about 60, 100, 600, 1000, 5,000, 10,000, 40,000, 100,000, or 400,000 different oligonucleotide probes per cm². The surface area of the array can be about or less than about 1, 1.6, 2, 3, 4, 5, 6, 7, 8, 9, or 10 cm².

Moreover, a person of ordinary skill in the art could readily analyze data generated using an array. Such protocols are disclosed above, and include information found in WO 9743450; WO 03023058; WO 03022421; WO 03029485; WO 03067217; WO 03066906; WO 03076928; WO 03093810; WO 03100448A1, all of which are specifically incorporated by reference.

2. Methylation

Any tests can be used to detect either hypermethylation, hypomethylation, or both. Suitable tests which can be used without limitation include lab-on-chip technology, microfluidic technologies, biomonitor technology, proton recognition technologies (e.g., Ion Torrent), and other highly parallel and/or deep sequencing methods. DNA hypemiethylation or hypomethylation may be used as indicators of gene silencing or gene activation, respectively. Once a biomarker is known as epigenetically silenced by DNA methylation, this signature is passed to daughter cells because of the molecular stability of this epigenetic modification. This stability is ideal for biomarker detection.

Epigenetic modification of a gene can be determined by any method known in the art. One method is to determine that a gene which is expressed in normal cells or other control cells is less expressed or not expressed in tumor cells, hypomethylation or silenced. This method does not, on its own, however, prove that the silencing or activation is epigenetic, as the mechanism of the silencing or inactivation could be genetic, for example, by somatic mutation. One method to determine that silencing is epigenetic is to treat with a reagent, such as DAC (5′-deazacytidine), or with a reagent which changes the histone acetylation status of cellular DNA or any other treatment affecting epigenetic mechanisms present in cells, and observe that the silencing is reversed, that the expression of the gene is reactivated or restored. Another means to determine epigenetic modification is to determine the presence of methylated CpG dinucleotide motifs in the silenced gene or the absence of methylation CpG dinucleotide motifs in the activated gene. Typically these methylated motifs reside near the transcription start site, for example, within about 3 kbp, within about 2.5 kbp, within about 2 kbp, within about 1.5 kbp, within about I kbp, within about 750 bp, or within about 500 bp. Once a gene has been identified as the target of epigenetic modification in tumor cells, determination of reduced or enhanced expression can be used as an indicator of epigenetic modification.

Expression of a gene can be assessed using any means known in the art. Typically expression is assessed and compared in test samples and control samples which may be normal, non-malignant cells. The test samples may contain cancer cells or pre-cancer cells or nucleic acids from them. Samples will desirably contain squamous Samples may contain mixtures of different types and stages of cancer cells. Either mRNA (or cDNA) or protein can be measured to detect expression which may be used as an indicator of epigenetic modification. Methods employing hybridization to nucleic acid probes can be employed for measuring specific mRNAs. Such methods include using nucleic acid probe arrays (microarray technology), in situ hybridization, and using Northern blots. Messenger RNA can also be assessed using amplification techniques, such as RT-PCR. Advances in genomic technologies now permit the simultaneous analysis of thousands of genes, although many are based on the same concept of specific probe-target hybridization. Sequencing-based methods are an alternative; these methods may be based on short tags, such as serial analysis of gene expression (SAGE) and massively parallel signature sequencing (MPSS). Differential display techniques provide yet another means of analyzing gene expression; this family of techniques is based on random amplification of cDNA fragments generated by restriction digestion, and bands that differ between two tissues identify cDNAs of interest.

Specific proteins can be assessed using any convenient method including immunoassays, immunohistochemistry, and immunocytochemistry but are not limited to that. Most such methods employ antibodies which are specific for the particular protein or protein fragments. The sequences of the mRNA (cDNA) and proteins of the markers of the present invention are known in the art and publicly available.

Methylation-sensitive restriction endonucleases can be used to detect methylated CpG dinucleotide motifs. Such endonucleases may either preferentially cleave methylated recognition sites relative to non-methylated recognition sites or preferentially cleave non-methylated relative to methylated recognition sites. Examples of the former are Acc Ban I, BstN I, Msp I, and Xma I. Examples of the latter are Acc II, Arm I, BssH II, BstU I, Hpa II, and Not I. Alternatively, chemical reagents can be used which selectively modify either the methylated or non-methylated form of CpG dinucleotide motifs.

Modified products can be detected directly, or after a further reaction which creates products which are easily distinguishable. Means which detect altered size and/or charge can be used to detect modified products, including but not limited to electrophoresis, chromatography, and mass spectrometry. Examples of such chemical reagents thr selective modification include hydrazine and bisulfite ions. Hydrazine-modified DNA can be treated with piperidine to cleave it. Bisulfite ion-treated DNA can be treated with alkali. Other means which rely on specific sequences can be used, including but not limited to hybridization, amplification, sequencing, and ligase chain reaction, Combinations of such techniques can be uses as is desired.

The principle behind electrophoresis is the separation of nucleic acids via their size and charge. Many assays exist for detecting methylation and most rely on determining the presence or absence of a specific nucleic acid product. Gel electrophoresis is commonly used in a laboratory for this purpose.

One may use MALDI mass spectrometry in combination with a methylation detection assay to observe the size of a nucleic acid product. The principle behind mass spectrometry is the ionizing of nucleic acids and separating them according to their mass to charge ratio. Similar to electrophoresis, one can use mass spectrometry to detect a specific nucleic acid that was created in an experiment to determine methylation.

One form of chromatography, high performance liquid chromatography, is used to separate components of a mixture based on a variety of chemical interactions between a substance being analyzed and a chromatography column. DNA is first treated with sodium bisulfite, which converts an umnethylated cytosine to uracil, while methylated cytosine residues remain unaffected. One may amplify the region containing potential methylation sites via PCR and separate the products via denaturing high performance liquid chromatography (DHPLC). DHPLC has the resolution capabilities to distinguish between methylated (containing cytosine) and unmethylated (containing uracil) DNA sequences.

Hybridization is a technique for detecting specific nucleic acid sequences that is based on the annealing of two complementary nucleic acid strands to form a double-stranded molecule. One example of the use of hybridization is a microarray assay to determine the methylation status of DNA. After sodium bisulfite treatment of DNA, which converts an unmethylated cytosine to uracil while methylated cytosine residues remain unaffected, oligonucleotides complementary to potential methylation sites can hybridize to the bisulfite-treated DNA. The oligonucleotides are designed to be complimentary to either sequence containing uracil (thymine) or sequence containing cytosine, representing unmethylated and methylated DNA, respectively. Computer-based microarray technology can determine which oligonucleotides hybridize with the DNA sequence and one can deduce the methylation status of the DNA. Similarly primers can be designed to be complimentary to either sequence containing uracil (thymine) or sequence containing cytosine. Primers and probes that recognize the converted methylated form of DNA are dubbed methylation-specific primers or probes (MSP).

An additional method of determining the results after sodium bisulfite treatment involves sequencing the DNA to directly observe any bisulfite-modifications. Pyrosequencing technology is a method of sequencing-by-synthesis in real time. It is based on an indirect bioluminometric assay of the pyrophosphate (PPi) that is released from each deoxynucleotide (dNTP) upon DNA-chain elongation. This method presents a DNA template-primer complex with a dNTP in the presence of an exonuclease-deficient Klenow DNA polymerase. The four nucleotides are sequentially added to the reaction mix in a predetermined order. If the nucleotide is complementary to the template base and thus incorporated, PPi is released. The PPi and other reagents are used as a substrate in a luciferase reaction producing visible light that is detected by either a luminometer or a charge-coupled device. The tight produced is proportional to the number of nucleotides added to the DNA primer and results in a peak indicating the number and type of nucleotide present in the form of a pyrogram. Pyrosequencing can exploit the sequence differences that arise following sodium bisulfite-conversion of DNA.

A variety of amplification techniques may be used in a reaction for creating distinguishable products. Some of these techniques employ PCR. Other suitable amplification methods include the ligase chain reaction (LCR) (Barringer et al., 1990), transcription amplification (Kwoh et al., 1989; WO88/10315), selective amplification of target polynucleotide sequences (U.S. Pat. No. 6,410,276), consensus sequence primed polymerase chain reaction (U.S. Pat. No, 4,437,975), arbitrarily primed polymerase chain reaction (WO90/06995), nucleic acid based sequence amplification (NASBA) (U.S. Pat. Nos. 5,409,818; 5,554,517; 6,063,603), microsatellite length polymorphism (MLP), and nick displacement amplification (WO2004/067726),

Sequence variation that reflects the methylation status at CpG dinucleotides in the original genomic. DNA offers two approaches to PCR primer design. In the first approach, the primers do not themselves “cover” or hybridize to any potential sites of DNA methylation; sequence variation at sites of differential methylation are located between the two primers. Such primers are used in bisulfite genomic sequencing, COBRA. Ms-SNuPE. In the second approach, the primers are designed to anneal specifically with either the methylated or unmethylated version of the converted sequence. If there is a sufficient region of complementarity, e.g., 12, 15, 18, or 20 nucleotides, to the target, then the primer may also contain additional nucleotide residues that do not interfere with hybridization but may be useful for other manipulations. Exemplary of such other residues may be sites for restriction endonuclease cleavage, for ligand binding or for factor binding or linkers or repeats. The oligonucleotide primers may or may not be such that they are specific for modified methylated residues

One way to distinguish between modified and unmodified DNA is to hybridize oligonucleotide primers which specifically bind to one form or the other of the DNA. After hybridization, an amplification reaction can be performed arid amplification products assayed. The presence of an amplification product indicates that a sample hybridized to the primer. The specificity of the primer indicates whether the DNA had been modified or not, which in turn indicates whether the DNA had been methylated or not. For example, bisulfite ions modify non-methylated cytosine bases, changing them to uracil bases. Uracil bases hybridize to adenine bases under hybridization conditions. Thus an oligonucleotide primer which comprises adenine bases in place of guanine bases would hybridize to the bisulfite-modified DNA, whereas an oligonucleotide Omer containing the guanine bases would hybridize to the non-modified (methylated) cytosine residues in the DNA. Amplification using a DNA polymerase and a second primer yield amplification products which can be readily observed. Such a method is termed MSP (Methylation Specific PCR; U.S. Pat. Nos. 5,786,146; 6,017,704; 6,200,756). The amplification products can be optionally hybridized to specific oligonucleotide probes which may also be specific for certain products. Alternatively, oligonucleotide probes can be used which will hybridize to amplification products from both modified and nonmodified DNA.

Another way to distinguish between modified and nonmodified DNA is to use oligonucleotide probes which may also be specific for certain products. Such probes can be hybridized directly to modified DNA or to amplification products of modified DNA. Oligonucleoti.d.e probes can be labeled using any detection system known in the art. These include but are not limited to fluorescent moieties, radioisotope labeled moieties, bioluminescent moieties, luminescent moieties, chemiluminescent moieties, enzymes, substrates, receptors, or ligands.

Still another way for the identification of methylated CpG dinucleotides utilizes the ability of the MBD domain of the McCP2 protein to selectively bind to methylated DNA sequences (Cross et al., 1994; Shiraishi et al., 1999). Restriction endonuclease digested genomic DNA is loaded onto expressed His-tagged methyl-CpG binding domain that is immobilized to a solid matrix and used for preparative column chromatography to isolate highly methylated DNA sequences.

Real time chemistry allows for the detection of PCR amplification during the early phases of the reactions, and makes quantitation of DNA and RNA easier and more precise. A few variations of the real-time PCR are known. They include the TaqMan™ (Roche Molecular Systems) system and Molecular BeaconTM system which have separate probes labeled with a fluorophore and a fluorescence quencher. In the Scorpion™ system the labeled probe in the form of a hairpin structure is linked to the primer. In addition, the Amplitluor™ (Chemicon International) system and the Plexor™ (Promega) system can be used.

DNA methylation analysis has been performed successfully with a number of techniques which include the MALDI-TOFF, Ma.ssARRAY, MethyLight, Quantitative analysis of ethylated alleles (QAMA), enzymatic regional methylation assay (ERMA), HeavyMethyl, QBSUPT, MS-SNuPE, MethylQuant, Quantitative PCR sequencing, and Oligonucleoti.d.e-based microarray systems.

The number of genes whose methylation is tested and/or detected can vary: one, two, three, four, five, six, seven, eight, nine, ten or eleven genes can be tested and/or detected. In some cases at least two genes are selected. In other embodiments at least three genes are selected.

Testing can be performed diagnostically or in conjunction with a therapeutic regimen. Testing can be used to monitor efficacy of a therapeutic regimen, for example, whether a chemotherapeutic agent or a biological agent, such as a polynucleotide. Testing can also he used to determine what therapeutic or preventive regimen to employ on a patient. Moreover, testing can be used to stratify patients into groups for testing agents and determining their efficacy on various groups of patients. Such uses characterize the cancer into categories based on the genes which are epigenetically silenced and/or the amount of silencing of the genes. In the case of a diagnosis or characterization, information comprising data or conclusions can be written or communicated electronically or orally. The identification may be assisted by a machine. Communication of the data or conclusions may be from a clinical laboratory to a clinical office, from a clinician to a patient, or from a specialist to a generalist, as examples. The form of communication of data or conclusions typically may involve a tangible medium or physical human acts.

A test sample obtainable from tissue or cell specimens or fluids includes detached tumor cells and/or free nucleic acids that are released from dead or damaged tumor cells. Nucleic acids include RNA, genomic DNA, mitochondrial DNA, single or double stranded, and protein-associated nucleic acids. Any nucleic acid specimen in purified or non-purified form obtained from such specimen cell can be utilized as the starting nucleic acid or acids. The test samples may contain cancer cells or pre-cancer cells or nucleic acids from them.

Demethylating agents can be contacted with cells in vitro or in vivo for the purpose of restoring normal gene expression to the cell or for validation of methylation. Suitable demethylating agents include, but are not limited to 5-aza-2′-deoxycytidine, 5-aza-cytidine, Zebularine, procaine, and L-ethionine. This reaction may be used for diagnosis, for determining predisposition, and for determining suitable therapeutic regimes.

Although diagnostic and prognostic accuracy and sensitivity may be achieved by using a combination of markers, such as 5 or 6 markers, or 9 or 10 markers, practical considerations may dictate use of smaller combinations. Any combination of markers for a specific cancer may be used which comprises 2, 3, 4, or 5 markers. Combinations of 2, 3, 4, or 5 markers can be readily envisioned given the specific disclosures of individual markers provided herein.

Kits according to the present invention are assemblies of reagents for testing methylation and/or silencing. They are typically in a package which contains all elements, optionally including instructions. Instructions may be in any form, including paper or digital. The instructions may be on the inside or the outside of the package. The instructions may be in the form of an interact address which provides the detailed manipulative or analytic techniques. The package may be divided so that components are not mixed until desired. Components may be in different physical states. For example, some components may be lyophilized and some in aqueous solution. Some may be frozen. Individual components may be separately packaged within the kit. The kit may contain reagents, as described above for differentially modifying methylated and non-methylated cytosine residues. Desirably the kit will contain oligonucleotide primers which specifically hybridize to regions within 1 kb of the transcription start sites of the selected genes/biomarkers. Additional markers may be used. Typically the kit will contain both a forward and a reverse primer for a single gene or marker. If there is a sufficient region of complementarily, e.g., 12, 15, 18, or 20 nucleotides, then the primer may also contain additional nucleotide residues that do not interfere with hybridization but may be useful for other manipulations. Exemplary of such other residues may be sites for restriction endonuclease cleavage, for ligand binding or for factor binding or linkers or repeats. The oligonucleotide primers may or may not be such that they are specific for modified methylated residues. The kit may optionally contain oligonucleotide probes. The probes may be specific for sequences containing modified methylated residues or for sequences containing non-methylated residues. The kit may optionally contain reagents for modifying methylated cytosine residues. The kit may also contain components for performing amplification, such as a DNA polymerase (particularly a thermostable DNA polymerase) and deoxyribonucleotides, labeled or not. Means of detection may also be provided in the kit, including detectable labels on primers or probes. Kits may also contain reagents for detecting gene expression. Such reagents may include probes, primers, or antibodies, for example. In the case of enzymes or ligands, substrates or binding partners may be used to assess the presence of the marker. Kits may contain 1, 2, 3, 4, or more of the primers or primer pairs of the invention. Kits that contain probes may have them as separate molecules or covalently linked to a primer for amplifying the region to which the probes hybridize. Other useful tools for performing the methods of the invention or associated testing, therapy, or calibration may also be included in the kits, including buffers, enzymes, gels, plates, detectable labels, vessels, etc. Kits may include tools for collecting suitable samples, such as tools for collecting oral swabs, oral biopsies, and endoscopes.

As an example, a gene may be contacted with hydrazine, which modifies cytosine residues, but not methylated cytosine residues. Then the hydrazine-treated gene sequence may be contacted with a reagent such as piperidine, which cleaves the nucleic acid molecule at hydrazine modified cytosine residues, thereby generating a product comprising fragments. By separating the fragments according to molecular weight, using, for example, an electrophoretic, chromatographic, or mass spectrographic method, and comparing the separation pattern with that of a similarly treated corresponding non-methylated gene sequence, gaps are apparent at positions in the test gene contained methylated cytosine residues. The presence of gaps is indicative of methylation of a cytosine residue in the CpG dinucleotide in the target gene of the test cell.

Bisulfite ions, for example, sodium bisulfite, convert non-methylated cytosine residues to bisulfite modified cytosine residues. The bisulfite ion treated gene sequence can be exposed to alkaline conditions, which convert bisulfite modified cytosine residues to uracil residues. Sodium hisulfite reacts readily with the 5,6-double bond of cytosine (but poorly with methylated cytosine) to form a sulfonated cytosine reaction intermediate that is susceptible to deamination, giving rise to a sulfonated uracil. The sulfonate group can be removed by exposure to alkaline conditions, resulting in the formation of uracil. The DNA can be amplified, for example, by PCR, and sequenced to determine whether CpG sites are methylated in the DNA of the sample. Uracil is recognized as a thymine by Tail polymerase and, upon PCR, the resultant product contains cytosine only at the position where 5-methylcytosine was present in the starting template DNA. One can compare the amount or distribution of uracil residues in the hisulfite ion treated gene sequence of the test cell with a similarly treated corresponding non-methylated gene sequence. A decrease in the amount or distribution of uracil residues in the gene from the test cell indicates methylation of cytosine residues in CpG dinucleotides in the gene of the test cell. The amount or distribution of uracil residues also can be detected by contacting the bisulfite ion treated target gene sequence, following exposure to alkaline conditions, with an oligonucleotide that selectively hybridizes to a nucleotide sequence of the target gene that either contains uracil residues or that lacks uracil residues, but not both, and detecting selective hybridization (or the absence thereof) of the oligonucleotide.

C. ALK inhibition

ALK inhibitors are potential anti-cancer drugs that act on tumors with variations of anaplastic lymphoma kinase (ALK) such as an EML4-ALK translocation. About 4-7% of non-small cell lung carcinomas (NSCLC) have EML4-ALK translocations, and others occur as well.

Approved inhibitors include crizotinib (Xalkori) and ceritinib (Zykadia), approved for treatment of NSCLC, and Alectinib (Alecensa) (Chugai. NDA has been filed in Japan) (breakthrough status in U.S.). Additional ALK inhibitors currently (or soon to be) undergoing clinical trials include Dalantercept, ACE-041 (Acceleron), Brigatinib (AP26113) (Ariad) (breakthrough status in U.S.) (also an EGFR inhibitor), Entrectinib (Nerviano's NMS-E628, licensed by Ignyta and renamed RXDX-101, in the U.S. orphan drug designation and rare pediatric disease designation for the treatment of neuroblastoma and orphan drug designation for treatment of TrkA-, TrkB-, TrkC-, ROSI- and ALK-positive NSCLC), PF-06463922 (Pfizer), TSR-Oil (Tesaro), CEP-37440 (Teva) and X-396 (Xcoveiy).

D. Drug Combinations

In the context of the present disclosure, it also is contemplated that ALK inhibitors described herein could be used similarly in conjunction with chemo- or radiotherapeutic intervention, or other treatments. It also may prove effective, in particular, to combine ALK inhibitors with other therapies that target different aspects of cancer cell function.

To kill cells, inhibit cell growth, inhibit metastasis, inhibit angiogenesis or otherwise reverse or reduce the malignant phenotype of tumor cells, using the methods and compositions of the present disclosure, one would generally contact a “target” cell with an interferon prodrugs according to the present disclosure and at least one other agent. These compositions would be provided in a combined amount effective to kill or inhibit proliferation of the cell. This process may involve contacting the cells with the interferon prodrugs according to the present disclosure and the other agents) or factor(s) at the same time. This may he achieved by contacting the cell with a single composition or pharmacological formulation that includes both agents, or by contacting the cell with two distinct compositions or formulations, at the same time, wherein one composition includes the interferon prodrugs according to the present disclosure and the other includes the other agent.

Alternatively, the ALK inhibitor therapy may precede or follow the other agent treatment by intervals ranging from minutes to weeks. In embodiments where the other agent and the interferon prodrugs are applied separately to the cell, one would generally ensure that a significant period of time did not expire between each delivery, such that the agent and expression construct would still be able to exert an advantageously combined effect on the cell. In such instances, it is contemplated that one would contact the cell with both modalities within about 12-24 hours of each other and, more preferably, within about 6-12 hours of each other, with a delay time of only about 12 hours being most preferred. In sonic situations, it may be desirable to extend the time period for treatment significantly, however, where several days (2, 3. 4, 5, 6 or 7) to several weeks (1, 2, 3, 4, 5, 6, 7 or 8) lapse between the respective administrations.

It also is conceivable that more than one administration of either interferon prodrugs or the other agent will be desired. Various combinations may be employed, where an ALK inhibitor according to the present disclosure is “A” and the other therapy is “B”, as exemplified below:

A/B/A B/A/B B/B/A A/A/B B/A/A A/B/B B/B/B/A B/B/A/B
A/A/B/B A/B/A/B A/B/B/A B/B/A/A B/A/B/A B/A/A/B B/B/B/A
A/A/A/B B/A/A/A A/B/A/A A/A/B/A A/B/B/B B/A/B/B B/B/A/B

Other combinations are contemplated. Again, to achieve cell killing, both agents are delivered to a cell in a combined amount effective to kill the cell.

Agents or factors suitable for cancer therapy include any chemical compound or treatment method that induces DNA damage when applied to a cell. Such agents and factors include radiation and waves that induce DNA damage such as, irradiation, microwaves, electronic emissions, and the like. A variety of chemical compounds, also described as “chemotherapeutic” or “genotoxic agents,” may be used. This may be achieved by irradiating the localized tumor site; alternatively, the tumor cells may be contacted with the agent by administering to the subject a therapeutically effective amount of a pharmaceutical composition.

Various classes of chemotherapeutic agents are contemplated for use with the present disclosure. For example, selective estrogen receptor antagonists (“SERMs”), such as Tamoxifen, 4-hydroxy Tamoxifen (Afimoxfene), Falsodex, Raloxifene, Bazedoxifene, Clomifene, Femarelle, Lasofoxifene, Ormeloxifene, and Toremifene.

Chemotherapeutic agents contemplated to be of use, include, e.g., camptothecin, actinomycin-D, mitomycin C. The disclosure also encompasses the use of a combination of one or more DNA damaging agents, whether radiation-based or actual compounds, such as the use of X-rays with cisplatin or the use of cisplatin with etoposide. The agent may be prepared and used as a combined therapeutic composition.

Heat shock protein 90 is a regulatory protein found in many eukaryotic cells. HSP90 inhibitors have been shown to be useful in the treatment of cancer. Such inhibitors include Geldanamycin, 17-(Allylamino)-17-demethoxygeldanamycin, PU-H71 and Rifabutin.

Agents that directly cross-link DNA or form adducts are also envisaged. Agents such as cisplatin, and other DNA alkylating agents may be used. Cisplatin has been widely used to treat cancer, with efficacious doses used in clinical applications of 20 mg/m² for 5 days every three weeks for a total of three courses. Cisplatin is not absorbed orally and must therefore be delivered via injection intravenously, subcutaneously, intratumorally or intraperitoneally.

Agents that damage DNA also include compounds that interfere with DNA replication, mitosis and chromosomal segregation. Such chemotherapeutic compounds include adriamycin, also known as doxorubicin, etoposide, verapamil, podophyllotoxin, and the like. Widely used in a clinical setting for the treatment of neoplasms, these compounds are administered through bolus injections intravenously at doses ranging from 25-75 mg/m² at 21 day intervals for doxorubicin, to 35-50 mg/m² for etoposide intravenously or double the intravenous dose orally. Microtubule inhibitors, such as taxanes, also are contemplated. These molecules are diterpenes produced by the plants of the genus Tacus, and include paclitaxel and docetaxel.

Epidermal growth factor receptor inhibitors, such as Iressa, mTOR, the mammalian target of rapamycin, also known as FK506-binding protein 12-rapamycin associated protein 1 (FRAP1) is a serine/threonine protein kinase that regulates cell growth, cell proliferation, cell motility, cell survival, protein synthesis, and transcription. Rapamycin and analogs thereof (“rapalogs”) are therefore contemplated for use in cancer therapy in accordance with the present disclosure. Another EGFR inhibitor of particular utility here is Gefitinib.

Another possible therapy is TNF-α (tumor necrosis factor-alpha), a cytokine involved in systemic inflammation and a member of a group of cytokines that stimulate the acute phase reaction. The primary role of TNF is in the regulation of immune cells. TNF is also able to induce apoptotic cell death, to induce inflammation, and to inhibit tumorigenesis and viral replication.

Agents that disrupt the synthesis and fidelity of nucleic acid precursors and subunits also lead to DNA damage. As such a number of nucleic acid precursors have been developed. Particularly useful are agents that have undergone extensive testing and are readily available. As such, agents such as 5-fluorouracil (5-FU), are preferentially used by neoplastic tissue, making this agent particularly useful for targeting to neoplastic cells. Although quite toxic, 5-FU, is applicable in a wide range of carriers, including topical, however intravenous administration with doses ranging from 3 to 15 mg/kg/day being commonly used.

Other factors that cause DNA damage and have been used extensively include what are commonly known as γ-rays, x-rays, and/or the directed delivery of radioisotopes to tumor cells. Other forms of DNA damaging factors are also contemplated such as microwaves and UV-irradiation. It is most likely that all of these factors effect a broad range of damage DNA, on the precursors of DNA, the replication and repair of DNA, and the assembly and maintenance of chromosomes. Dosage ranges for x-rays range from daily doses of 50 to 200 roentgens for prolonged periods of time (3 to 4 weeks), to single doses of 2000 to 6000 roentgens. Dosage ranges for radioisotopes vary widely, and depend on the half-life of the isotope, the strength and type of radiation emitted, and the uptake by the neoplastic cells.

In addition, it also is contemplated that immunotherapy, hormone therapy, toxin therapy and surgery can be used.

The skilled artisan is directed to “Remington's Pharmaceutical Sciences” 15th Edition, Chapter 33, in particular pages 624-652. Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

### III. PHARMACEUTICAL COMPOSITIONS AND DELIVERY METHODS

Where clinical applications are contemplated, pharmaceutical compositions will be prepared in a form appropriate for the intended application. Generally, this will entail preparing compositions that are essentially free of pyrogens, as well as other impurities that could be harmful to humans or animals.

One will generally desire to employ appropriate salts and buffers to render drugs stable and allow for uptake by target cells. Aqueous compositions of the present invention comprise an effective amount of the drug dissolved or dispersed in a pharmaceutically acceptable carrier or aqueous medium. The phrase “pharmaceutically or pharmacologically acceptable” refer to molecular entities and compositions that do not produce adverse, allergic, or other untoward reactions when administered to an animal or a human. As used herein, “pharmaceutically acceptable carrier” includes solvents, buffers, solutions, dispersion media, coatings, antibacterial and antifungal agents, isotonic and absorption delaying agents and the like acceptable for use in formulating pharmaceuticals, such as pharmaceuticals suitable for administration to humans. The use of such media and agents for pharmaceutically active substances is well known in the art. Except insofar as any conventional media or agent is incompatible with the active ingredients of the present invention, its use in therapeutic compositions is contemplated. Supplementary active ingredients also can be incorporated into the compositions, provided they do not inactivate the vectors or cells of the compositions.

The active compositions of the present invention may include classic pharmaceutical preparations. Administration of these compositions according to the present invention may be via any common route so long as the target tissue is available via that route, but generally including systemic administration. This includes oral, nasal, or buccal. Alternatively, administration may be by intradermal, subcutaneous, intramuscular, intraperitoneal or intravenous injection, or by direct injection into muscle tissue. Such compositions would normally be administered as pharmaceutically acceptable compositions, as described supra.

The active compounds may also be administered parenterally or intraperitoneally. By way of illustration, solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations generally contain a preservative to prevent the growth of microorganisms.

The pharmaceutical thnns suitable for injectable use include, for example, sterile aqueous solutions or dispersions and sterile powders for the extemporaneous preparation of sterile injectable solutions or dispersions. Generally, these preparations are sterile and fluid to the extent that easy injectability exists. Preparations should be stable under the conditions of manufacture and storage and should be preserved against the contaminating action of microorganisms, such as bacteria and fungi. Appropriate solvents or dispersion media may contain, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial an antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions may be prepared by incorporating the active compounds in an appropriate amount into a solvent along with any other ingredients (for example as enumerated above) as desired, followed by filtered sterilization. Generally, dispersions are prepared by incorporating the various sterilized active ingredients into a sterile vehicle which contains the basic dispersion medium and the desired other ingredients, e.g., as enumerated above, in the case of sterile powders for the preparation of sterile injectable solutions, the preferred methods of preparation include vacuum-drying and freeze-drying techniques which yield a powder of the active ingredient(s) plus any additional desired ingredient from a previously sterile-filtered solution thereof.

The compositions of the present invention generally may be formulated in a neutral or salt form. Pharmaceutically-acceptable salts include, for example, acid addition salts (formed with the free amino groups of the protein) derived from inorganic acids (e.g., hydrochloric or phosphoric acids, or from organic acids (e.g., acetic, oxalic, tartaric, mandelic, and the like.

Salts formed with the free carboxyl groups of the protein can also be derived from inorganic bases (e.g., sodium, potassium, ammonium, calcium, or ferric hydroxides) or from organic bases (e.g., isopropylamine, trimethylamine, histidine, procaine and the like.

Upon formulation, solutions are preferably administered in a manner compatible with the dosage formulation and in such amount as is therapeutically effective. The formulations may easily be administered in a variety of dosage forms such as injectable solutions, drug release capsules and the like. For parenteral administration in an aqueous solution, for example, the solution generally is suitably buffered and the liquid diluent first rendered isotonic for example with sufficient saline or glucose. Such aqueous solutions may be used, for example, for intravenous, intramuscular, subcutaneous and intraperitoneal administration.

Preferably, sterile aqueous media are employed as is known to those of skill in the art, particularly in light of the present disclosure. By way of illustration, a single dose may be dissolved in 1 ml of isotonic NaCl solution and either added to 1000 ml of hypodennoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

### IV. EXAMPLES

The following examples are included to demonstrate preferred embodiments of the disclosure. It should he appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1

### Materials and Methods

Cells. Human OSCC cell lines, OSCC cell lines, HSC3 and Ca127 were derived from human primary tongue OSCC. Ca127 cells were obtained from ATCC (Rockville, Md.), HSC3 cells were kindly provided by Dr. Brian Schmidt (NYU) (Saghafi et al., 2011) and authenticated by G netica DNA Laboratories (Cincinnati, Ohio). Cells were maintained in DMEM (Gibco, Carlsbad, Calif.) containing 10% FBS at 37 ° C. in 5% CO₂.

Reagents. Gefitinib (EGFR inhibitor) and TA.E684 (ALK inhibitor) were obtained from Selleck Chemicals (Houston, Tex.). TAE684 (50 mg/ml) and Gefitinib (5 mg/ml) were used for OSCC cell treatments in vitro. For animal studies, the inventors used TAE684 (10 mg/kg) and Gefitinib (100 mg/kg) dissolved in 0.05% propylene glycol.

Immunohistochemical (IHC) staining. Formalin fixed paraffin-embedded (FFPE) specimens (n=3 per group) of HSC3-derived xenografts and human OSCC tissue arrays, containing stage I (TIN0M0) and stage IV (T4N0M0) OSCC biopsied from the tongue and normal tongue epithelium, were de-paraffinized, rehydrated, and blocked with 3% normal goat serum. Immunohistochemistry was performed using the Vectastain Elite ABC Kit (Vector Laboratories, Burlingame, Calif.) according to manufacturer's protocol. Primary antibodies to p-ALK (1:100; MBS855239; MyBioSource; San Diego, Calif.) were used. Secondary anti-rabbit antibody Substrate interactions were visualized with DAB (Vector Laboratories, Burlingame, Calif.). Negative controls were incubated in pre-immune serum alone. IHC staining quantification was made using three non-overlapping fields per specimen, selected indiscriminately for analysis. Measurements were made with the use of NIS-Elements imaging software (Nikon, Brighton, Mich.) associated with a Nikon TE200U microscope and quantified using MU Image) (v1.49) (rsb.info.nih.gov/ij) as previously described Mustafa et at. (2015). Briefly, the optical density (OD) of p-ALK immunostaining was evaluated with OD estimated using the following formula:

OD log(max intensity/mean intensity), where max intensity=255 for 8-bit images

### See Mustafa et al. (2015).

Immunolluorescent (IF) staining. OSCC cells were cultured on coverslips (1×10⁵), fixed with 4% paraformaldehyde and stained as previously described (Siddiqa et al., 2012; Jeske et at., 2011) with antibodies to total ALK antibody (1:100; #ABN263 EMD Millipore, Billerica, Mass.) and p-ALK (1:100); #MBS855239; MyBio-Source; San Diego, Calif.) and anti-rabbit Alexa Flour 488 secondary antibody (1:100; #A-11008, ThermoFisher, Waltham, Mass.) and DAPI 1 μg/ml (ThermoFisher) as a nuclear stain. Images were acquired with a Nikon TE2000U microscope using a 40×/1.3NA objective lens and processed for illustration purposes with NIS-Elements imaging software (Nikon, Brighton, Mich.).

Cell viability assays. Cytotoxicity was assessed using the Cell Titer 96 Aqueous Non-Radioactive Cell Proliferation Assay (Promega, Madison, Wis.) according to manufacturer's protocol. Absorbance values of test groups were normalized against controls (n=4).

Cell migration assay. Cellular migration was performed using the IncuCyte ZOOM™ live cell image device (Essen Bioscience). After achieving cell confluence in 96-well format, the IncuCyte scratch system was used to generate simultaneous rectangular ‘wounds’ with a defined area in HSC3 and Ca127 cell layers. Cells were then subjected to treatment with Gefitinib and/or TAE684 (concentrations specified above) for the time course indicated in the figures. Images of cells moving within the wound area were taken by the automated Incu-Cyte system over intervals of 2 hr (HSC3) or 3 hr (Ca127). Migration was calculated as percent of cell confluence within the wound, starting at 0% confluence (i.e., no cells in the wound area). When the wound was completely filled with migrating cells, this was calculated at 100% confluence.

Flow cytometry. OSCC cell lines were cultured to 50% confluency and treated with Gefitinib (500 nM), TAE684 (500 nM) and combination treatments for 30 hr. Cells were harvested, fixed in 70% EtoH, treated with RNase A, and stained with propidium iodide (PI). FACS analysis of DNA profiles were performed to quantitate cells in the sub-G1, G1, S+G2/M phases of the cell cycle.

Immunoblotting. OSCC cell lines treated with vehicle, Gefitinib (500 nM) and/or TAE684 (500 nM) for 6 h were harvested and lysed in 1% Triton-PBS. Cell lysates (100 units/A280) were used for protein electrophoresis in 10% SDS-PAGE. SDS-PAGE separated proteins were transferred to PVDF membrane and the membrane blocked in 5% milk. Rabbit monoclonal antibodies against STAT3 (1:2000; #12640), p-STAT3(Tyr705) (1:4000; #9145) and ERK1/2 (1:2000; #4695) were used (Cell Signaling; Danvers, Mass.). Rabbit polyclonal antibodies against AKT (1:2000; #9272), p-AKT(Ser473) (1:4000; #9271), EGFR (1:1000, #4267), p-EGFR(Y1148) (1:4000; #4404) and p-ERKI/2 (1:5000; #9101) were also used (Cell Signaling; Danvers, Mass.). GAPDH rabbit polyclonal antibodies (1:1000; Rockland. #600-401-A33, Limerick, Pa.) were used to confirm equal loading of protein, Primary antibodies were diluted in a total of 5 ml diluent (1% milk in PBS-0.1% Tw-20) and the membrane incubated overnight at 4° C. The membrane was washed 3 with PBS-Tw-20, incubated with ECL Plus detection solution (GE Healthcare, South San Francisco, Calif.) for 1 min and signal detected by exposure to radiograph film for 30 sec. Band intensities were quantified using ImageJ software.

OSCC mouse xenograft models. All studies were approved by the UTHSCSA institutional Animal Care and Use Committee. Six week-old female athymic nude mice (Harlan, Indianapolis, Tenn.) were used in a laminar air-flow cabinet under pathogen-free conditions. Mice were provided with a 12 h light/dark schedule at controlled temperature and humidity with food and water ad libitum. Mice were acclimated for one week prior to study initiation. Mice were injected subcutaneously in the right flank with 3×10⁶ HSC3 cells in 0.1 ml of sterile PBS. Two weeks postinoculation, tumors grew to an average volume of 125 mm³. Mice were stratified into four experimental groups (n=5 per group), which received the following treatments via oral gavage: group A, vehicle control (170 μl); group B, TAE684 (10 mg/kg; 170 μl); group C, Gefitinib (100 mg/kg; 170 μl), and group D, TAE684 (10 mg/kg)+Gefitinib (100 mg/kg); 170 μl). Treatments were repeated every day for a total of 14 days. Mice were monitored daily for tumor growth (using digital calipers), cachexia, and weight loss. Tumor volumes were calculated by the elliptical formula: ½ (Length×Width²) (Jensen et al., 2008). At experimental conclusion, tumors were fixed and processed for histological analysis. Hematoxylin and eosin (H&E) staining and IHC studies were performed by the UTHSCSA Cancer Therapy and Research Center (CTRC) core pathology laboratory.

Statistical analysis. Statistical analysis was performed using GraphPad Prism4 (San Diego, Calif.). Cell viability assays (n=4 per group), cell migration assays (n=4 per group), Western blot band intensities (n=3 per group), and IHC staining (n=3 per group) were analyzed by one-way ANOVA and Bonferroni's post hoc test Statistical analyses of tumor growth were made using analysis of variance with repeated measures with Bonferroni's post hoc test (n=5 per group). A p value less than 0.05 was considered statistically significant.

### Example 2

### Results

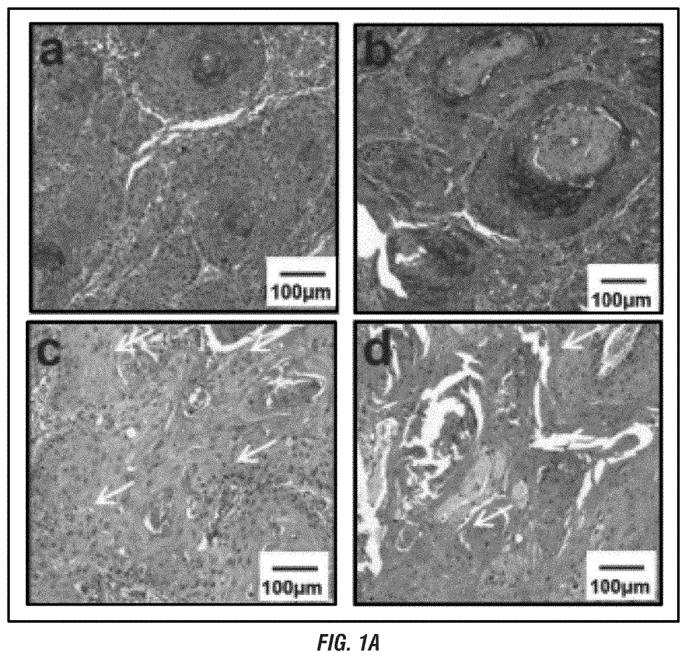

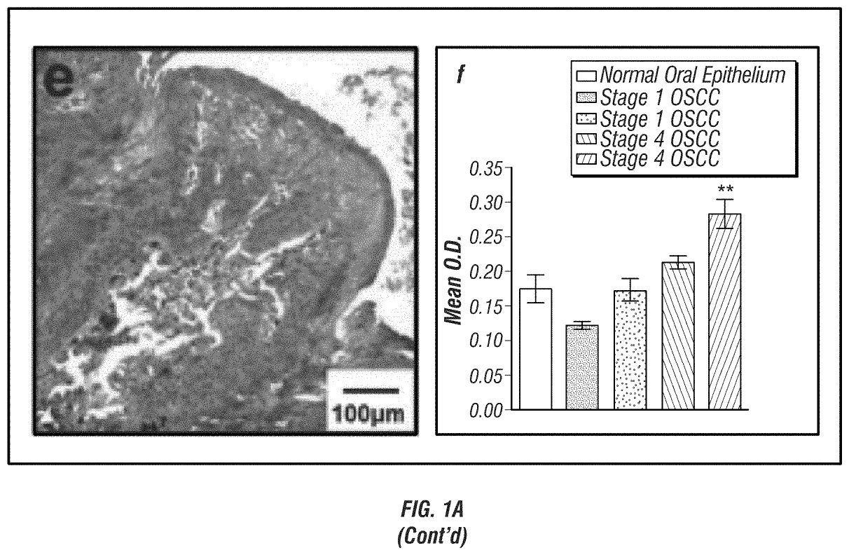

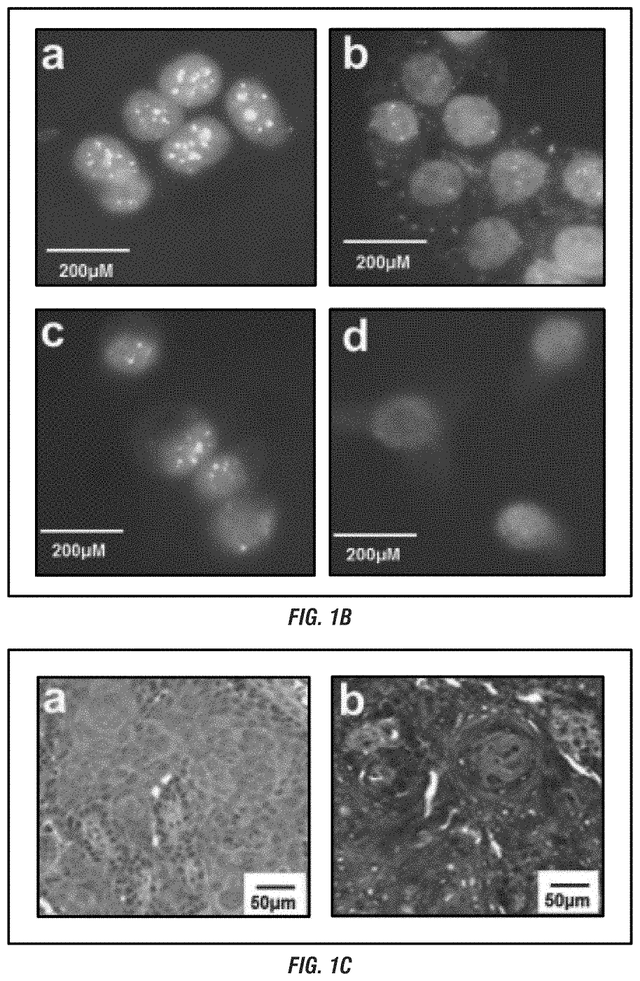

Activated ALK is expressed in late-stage human OSCC and in OSCC cell lines/xenografts. The inventors previous studies showed differential expression in ALK was associated with late-stage OSCC (Huang et al., 2013). They also showed that OSCC cell lines exhibit different levels of ALK mRNA expression, which corresponded with cell invasiveness (Huang et al., 2013). To further examine the role of ALK in OSCC, the inventors examined expression of activated ALK (the phosphorylated form, phospho-ALK) in a human OSCC tissues containing early-stage (stage 1) and late-stage (stage 4) OSCCs and in OSCC cell lines. HSC3 and Ca127. IHC analysis of human OSCC showed that activated phospho-ALK was expressed at relatively high levels in stage 4 OSCCs (FIG. 1A, panels a, b, & f; p<0.01), whereas staining for phospho-ALK was negative in early-stage OSCC (FIG. 1A, panels c, d & f) and normal control (FIG. 1A, panel c). Non-specific staining in normal oral epithelium is due to tissue folding at the periphery. Immunofluorescent staining revealed expression of total and activated ALK in both OSCC cell lines (FIG. 1B). Total ALK exhibited membrane and cytoplasmic staining as well as more prominent nuclear staining (FIG. 1B, panels a & c). Activated phospho-ALK demonstrated mainly membrane/cytoplasmic localization with some nuclear staining (FIG. 1B, panels b & d). This immunostaining analysis revealed that HSC3-derived tumors also express high levels of activated phospho-ALK in vivo (FIG. 1C, panel b).

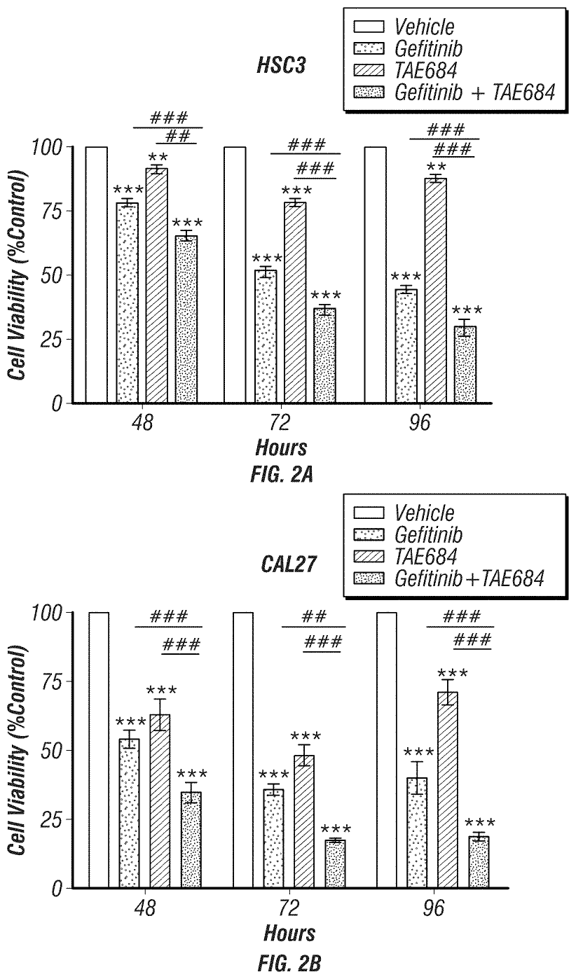

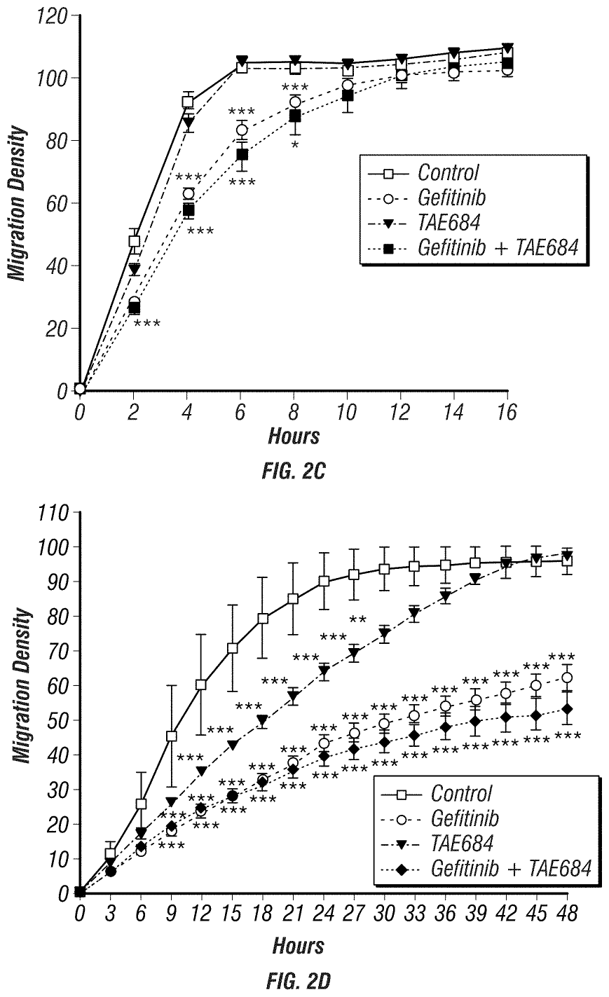

Co-targeting ALK and EGFR has additive effects on cell growth in OSCC cell lines. Because ALK was previously shown to interact with the EGFR pathway, which may explain the poor response of OSCCs to drugs targeting EGFR, the inventors examined the response of HSC3 and Ca127, two OSCC cell lines with high ALK expression and activity, to inhibitors of ALK and EGFR singly and in combination. Based upon preliminary data from dose response curves (data not shown), OSCC cell lines were treated with 500 nM of the ALK inhibitor TAE684, 500 nM of the EGFR inhibitor Gefitinib, and a combination of both for 48, 72. and 96 h. A progressive decrease of OSCC cell growth was observed over a 96 h time course with these treatments (FIGS. 2A and B). HSC3 and Ca127 cells demonstrated significant anti-proliferative effects in response to single anti-ALK or anti-EGFR treatments in vitro. Co-treatment with Gefitinib and TAE684 resulted in a significant additive reduction in cell growth at all time-points (p<0.001). To determine whether decrease in cellular proliferation was due to cell cycle effects, the inventors performed flow cytometry cell staining with P1. These data suggest that cell cycle arrest at the G1 phase associated with a decrease in S phase and an increase in the SubG1 phase may, at least in part,account for decreased proliferation of HSC3 and Ca127 in single treatments (Tables 1 and 2).

The inventors then examined the effects of ALK and/or EGFR inhibition on HSC3 and Ca127 cell migration using the IncuCyte in vitro ‘wound’ healing assay. HSC3 and Ca127 filled the ‘wound’ (i.e., reaching 100% migration density) within 6 and 24 h, respectively, after the scratch (Huang et al., 2013). Mobility was significantly reduced with EGFR inhibition in both cell lines. The highly invasive HSC3 cells displayed significant reduction from 2 to 8 h and completely filling the wound by 10 h (p<0.001, FIG. 2C). In contrast, migration of the less invasive Ca127 cells was significantly reduced at 9 h and remained significantly low for 48 h (FIG. 2D; p<0.001). Co-treatment of both HSC3 and Ca127 cells resulted in greater reduction in cell migration than the effects seen with Gefitininb or TAE684 alone (FIGS. 2C and 2D; p<0.001); however the difference between the combination treatments and single treatments with Gefitinib was not statistically significant.

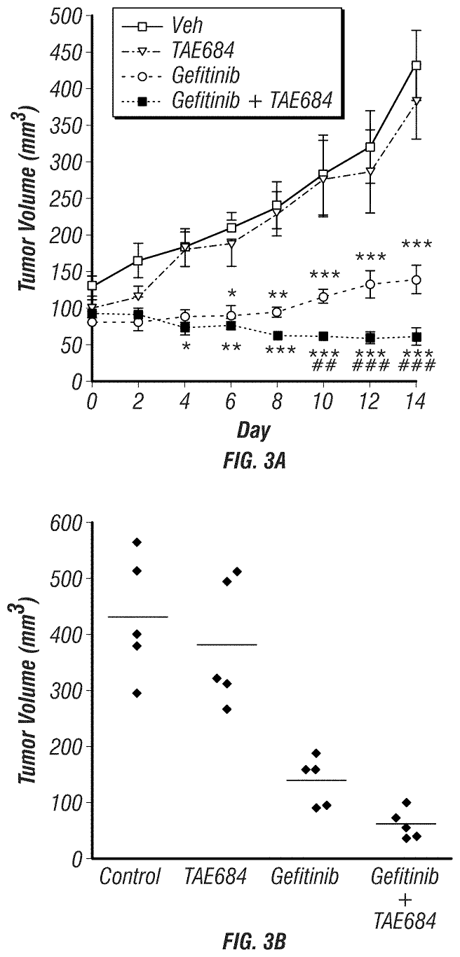

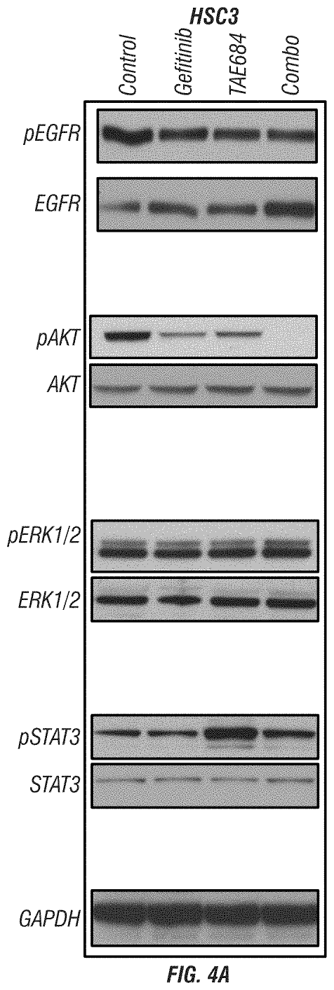

Co-targeting ALK and EGFR has additive effects on tumor growth in vivo. To further investigate the effects of inhibiting ALK and EGFR in vivo, the inventors generated mouse OSCC xenografts using HSC3 cells (FIG. 3A). The findings in this model recapitulated the in vitro studies in that HSC3-derived tumor growth was significantly reduced by EGFR inhibition alone (57% of control) as early as day 6 and co-treatments at day 4 (35% of control) (FIG. 3A). Notably, HSC3-derived tumor growth was unaffected by single anti-ALK treatments. However, co-treating with Gefitinib and TAE684 further reduced tumor growth (p<0.001) and, in fact, caused a significant and dramatic shrinkage in tumor volumes by day 14 with mean volumes of 60 mm³ (co-treatment), versus 431 mm³ (vehicle control), 381 mm3 (TAE684), and 138 mm³ (Gefitinib). The effects of co-treating with Gefitinib and TAE684 were statistically significantly compared to single Gefitinib treatment, suggesting that while ALK inhibition alone is not efficacious, it does enhance the anti-tumorigenic effects of the EGFR inhibitor (p<0.001; FIG. 3A).

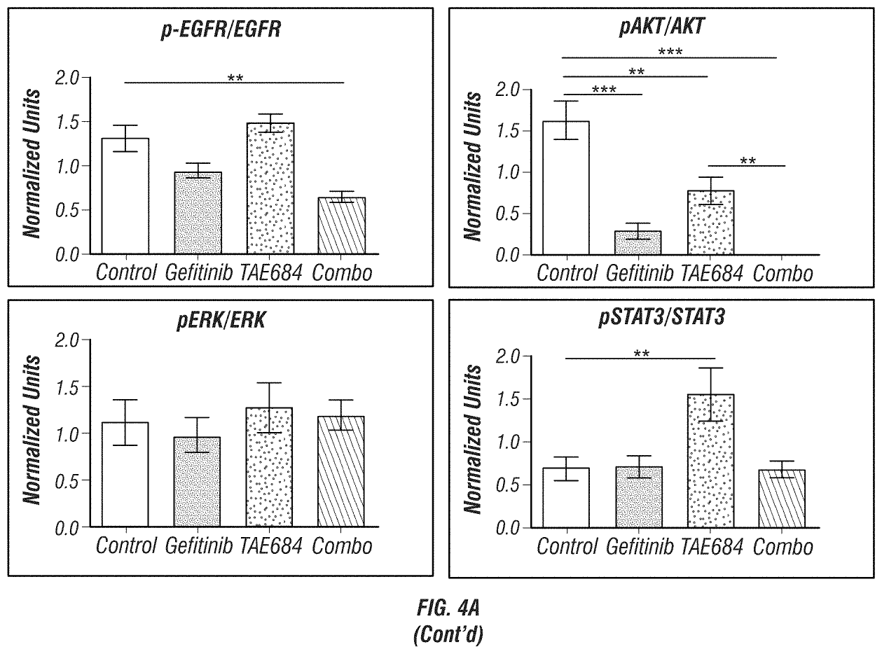

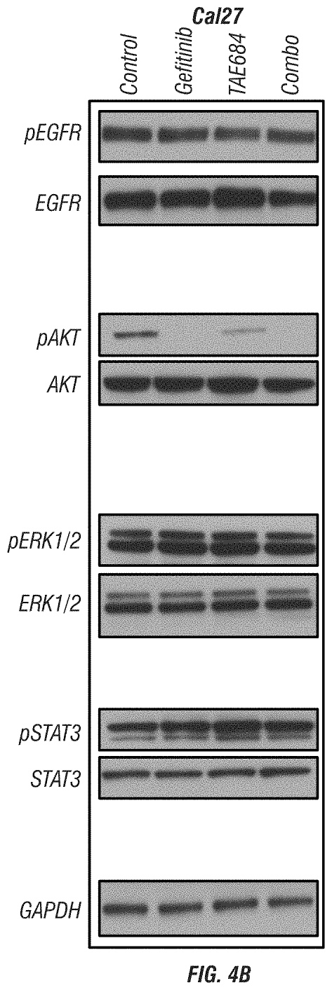

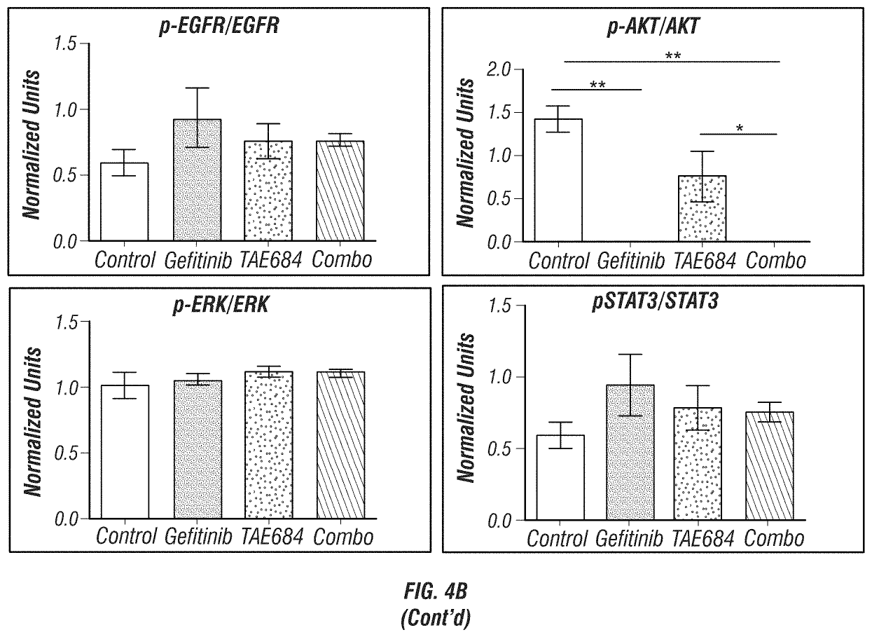

Targeting ALK and EGFR parallel signaling pathways in OSCC cells abolishes AKT signaling, in vitro. To investigate the molecular mechanisms behind parallel signaling between ALK and EGFR in OSCC, the inventors examined the effects of ALK and/or EGFR inhibition on the activity of common cellular signaling pathways, using Western blot analysis of HSC3 and Ca127 cell lines. Both Gefitinib and TAE684 significantly reduced AKT activation in HSC3 cells; however co-treatments completely abolished phospho-AKT (FIG. 4A). Ca127 cells treated with Gefitinib alone and in combination with TAE684 also demonstrated undetectable levels of phospho-AKT (FIG. 4B). TAE684 treatment significantly decreased phospho-AKT in both OSCC cell lines but seemed to be less effective than Gefitinib. Levels of total and phospho-ERK1/2, another downstream effector of EGFR and ALK, were unaffected by any of the treatments. Notably, activation of STAT-3 was induced by TAE684 treatment in HSC3 cells (p<0.01) and slightly induced in Ca127 Taken together, EGFR and ALK inhibitors effects on these parallel pathways appear to converge upon AKT signaling in OSCC cell lines.

TABLE 1
Cal27 cell cycle and analysis.

| Treatment | SubG1 | G1 | S | G2 |
|---|---|---|---|---|
| Control | 0.95 | 86.78 | 5.12 | 7.15 |
| Gefitinib | 2.53 | 90.79 | 2.59 | 4.44 |
| TAE684 | 1.68 | 88.11 | 2.25 | 8.40 |
| Combination | 1.94 | 88.7 | 2.32 | 7.65 |

Values represent % of cell distribution.

TABLE 2
HSC3 cell cycle analysis.

| Treatment | SubG1 | G1 | S | G2 |
|---|---|---|---|---|
| Control | 0.84 | 85.67 | 8.21 | 4.91 |
| Gefitinib | 2.03 | 95.60 | 0.0 | 1.87 |
| TAE684 | 0.34 | 95.23 | 0.84 | 3.73 |
| Combination | 1.90 | 96.25 | 0.59 | 2.26 |

Values represent % of cell distribution.

### Example 3

### Discussion

The invasion of OSCC into loco-regional structures is a deadly consequence for patients with this malignancy. As of yet, effective therapies that target invasive OSCC are lacking. Although EGFR is expressed in the majority of OSCCs, drugs targeting this tyrosine kinase receptor have surprisingly led to little effect. Although this might be due to increased copy number of EGFR, which is associated with poor prognosis in head and neck cancer, studies showed that EGFR gene amplification, polysotny and truncation (EGFRvIII) do not predict response to EGFR inhibitors (Chung et al., 2006; Fukuoka et al., 2011; Szabo et al., 2011). Thus, the reasons behind the lack of efficacy of EGFR targeted therapy in OSCC remain unclear.

The inventors' analysis of EGFR RNA expression detected the full receptor, and to a much lower extent the truncated variant EGFRvIII (data not shown); correspondingly, protein expression of full EGFR (FIGS. 4A-B) was detected but not EGFRvIII (data not shown; antibody used to internal EGFR domain can detect both full and truncated EGFR based on differences in size), suggesting that EGFRvIII likely does not play a role in decreased EGFR inhibitor efficacy. Indeed, several reports have indicated that this mutation is not predictive of response to EGFR-targeted therapies.

Another mechanism that may account for the lack of efficacy of EGFR inhibitors is the activity of compensating pathways that may diminish efficacy of these drugs. The inventors have previously shown that ALK may be induced in OSCC, and ALK activity may lead to enhanced invasiveness in OSCC (Huang et al., 2013). In this study, the inventors examined the interaction between ALK and EGFR in OSCC. This was based on the evidence that induction of EGFR signaling confers resistance to anti-ALK treatment in NSCLC and that anti-EGFR treatment is not effective in OSCC. The inventors hypothesized that co-targeting ALK and EGFR would lead to enhanced anti-oncogenic response compared to single treatments. To pursue this hypothesis, the inventors tested the effects of ALK and EGFR inhibitors, TAE684 and Gefitinib, respectively, on two OSCC cell lines HSC3 and Ca127 expressing both ALK and EGFR.

The inventors in vitro and in vivo assays using HSC3 and Ca127 cells reveal that while single treatments against EGFR or ALK have varying effects on growth, combination treatments, on the other hand, were very effective in significantly reducing growth in vitro and in viva. While decrease in proliferation from single treatments may be in part due to arrest at the G phase, apoptotic effects cannot be ruled out. By examining downstream pathways, including AKT, ERK1/2, and STAT3, the inventors found that AKT may be the mechanistic target in combination treatments. Interestingly, while AKT activity was diminished by EGFR inhibition in Ca127, the effect was less prominent in HSC3 cells. Nonetheless, co-treatment with ALK and EGFR inhibitors resulted in equivalent effect in abolishing activated levels of AKT in both HSC3 and Ca127 cells. Also, Ca127 was more responsive to decreased growth by ALK inhibition than HSC3 cells, which was consistent with a stronger response in decreasing AKT activity, suggesting that AKT regulation by ALK may modulate aggressive growth of OSCC.

Further studies are required to understand the regulation of AKT by ALK and their downstream role in OSCC growth and invasion. Importantly, the data highlight that the AKT pathway may be a downstream therapeutic target that modulates both ALK and EGFR activity in OSCC. Previous studies suggest that phosphorylation of AKT predicts poor clinical outcomes in OSCC and significantly correlates with local recurrences and low five-year survival rates (Yu et of., 2007, Li et al., 2013). AKT activation is also shown to be a significant prognostic indicator for OSCC with lymph node metastasis (Knowles et al., 2011). Several PI3/AKT pathway inhibitors have been tested in preclinical and clinical trials, which may provide new avenues for the use of these drugs in the treatment of OSCC (Pal & Handal, 2012). While the inventors' data suggest that anti-AKT drugs may be a therapeutic strategy in the treatment of OSCC, further examination of this pathway in invasive OSCC may lead to a more efficacious and targeted therapeutic intervention that may also include combinations of EGFR or ALK inhibitors.

Another factor that may affect efficacy of response to receptor tyrosine kinase receptor inhibitors is the translocation of these receptors into the nucleus (Wang & Hung, 2012). Previous studies have shown that EGFR and receptor family member ErbB2 translocate into the nucleus where they act as transcription factors (Wang & Hung, 2012). Similarly ALK has been shown to he present in the nucleus, which has so far been associated with the NPM-ALK fusion but not the wild-type form (Duyster et al., 2001). In the case of EGFR and ERbB2, full receptors (no fusions) are transported to the nucleus via endocytosis (Wang & Hung, 2012). It is yet to be seen if this is also true for ALK. Our IF data suggest that full length ALK is translocated in the nucleus in HSC3 and Ca127 OSCC cells, which raises the possibility that it could exert transcriptional effects in addition to the cell signaling effects described above. Interestingly, ErbB2 nuclear interaction with STAT3 and actions of this complex as a transcription factor was observed in human breast cancer cells (Wang & Hung, 2012). Additional studies are needed to examine the mechanisms underlying the translocation of ALK and consequent interactions at the nuclear level and regulation of STAT3 at the signal transduction level.

In summary, these findings suggest that co-targeting ALK and EGFR signaling may enhance the efficacy of EGFR targeted therapies. Treatment with bath the ALK and EGFR inhibitors led to additive effects in OSCC cells exhibiting elevated ALK activity. AKT may he a converging point for ALK and EGFR signaling, and STAT3 may modulate ALK activity. Additional studies are required to examine the role of AKT as an effector of ALK and EGFR and the potential of its utility as a therapeutic target alone or in combination K and/or EGFR inhibitors.

All of the compositions and/or methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions and/or methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would he achieved. All such similar substitutes and modifications apparent to those skilled in the art are deemed to be within the spirit, scope and concept of the disclosure as defined by the appended claims.

### VI. REFERENCES

The following references, to the extent that they provide exemplary procedural or other details supplementary to those set forth herein, are specifically incorporated herein by reference.
European Appln. 320 308
European Appln. 329 822
European Appln. 373 203
European Appln. 785 280
European Appln. 799 897
GB Appln. 2 202 328
PCT Appln. PCT/US87100880
PCT Appln. PCT/US89101025
PCT Appln. WO 88/10315
PCT Appln. WO 89/06700
PCT Appln. WO 90/07641
PCT Appln. WO 93/17126
PCT Appln, WO 95./11995
PCT Appln, WO 95/21265
PCT Appln. WO 95/21944
PCT Appln. WO 95/35505
PCT Appln. WO 96/31622
PCT Appln. WO 97/10365
PCT Appln. WO 97/27317
PCT Appln. WO 99/35505
U.S. Pat. No. 3,817,837
U.S. Pat. No. 3.850.752
U,S. Pat. No. 3,939,350
U.S. Pat. No. 3,996,345
U.S. Pat. No. 4,277,437
U.S. Pat. No. 4,275,149
U.S. Pat. No. 4,366,241
U.S. Pat. No. 4,683,195
U.S. Pat. No. 4,683,202
U.S. Pat. No. 4,800,159
U.S. Pat. No. 4,883,750
U.S. Pat. No. 5,143,854
U.S. Pat. No. 5,202,231
U.S. Pat. No. 5,242,974
U.S. Pat. No. 5,279,721
U.S. Pat. No. 5.288.644
U.S. Pat. No. 5,319,080
U.S. Pat. No. 5,324,633
U.S. Pat. No. 5,384,261
U.S. Pat. No. 5,405,783
U.S. Pat. No. 5,412,087
U.S. Pat. No. 5,424,186
U.S. Pat. No. 5,429,807
U.S. Pat. No. 5,432,049
U.S. Pat. No. 5,436,327
U.S. Pat. No. 5.445.934
U.S. Pat. No. 5,468,613
U.S. Pat. No. 5,470,710
U.S. Pat. No. 5,472,672
U.S. Pat. No. 5,492,806
U.S. Pat. No. 5,503,980
U.S. Pat. No. 5,510,270
U.S. Pat. No. 5,514,785
U.S. Pat. No. 5,519,134
U.S. Pat. No. 5,525,464
U.S. Pat. No. 5,527,681
U.S. Pat. No. 5,529,756
U.S. Pat. No. 5,532,128
U.S. Pat. No. 5,545,531
U.S. Pat. No. 5,547,839
U.S. Pat. No. 5,554,501
U.S. Pat. No. 5,556,752
U.S. Pat. No. 5,561,071
U.S. Pat. No. 5,567,811
U.S. Pat. No. 5.571.639
U.S. Pat. No. 5,576,427
U.S. Pat. No. 5,580,726
U.S. Pat. No. 5,580,732
U.S. Pat. No. 5,593,839
U.S. Pat. No. 5,599,672
U.S. Pat. No. 5,599,695
U.S. Pat. No. 5,610,287
U.S. Pat. No. 5,624,711
U.S. Pat. No. 5,631,134
U.S. Pat. No. 5,639,603
U.S. Pat. No. 5.654.413
U.S. Pat. No. 5,658,734
U.S. Pat. No. 5,661,028
U.S. Pat. No. 5,665,547
U.S. Pat. No. 5,667,972
U.S. Pat. No. 5,695,940
U.S. Pat. No. 5,700,637
U.S. Pat. No. 5,744,305
U.S. Pat. No. 5,757,994
U.S. Pat. No. 5,788,166
U.S. Pat. No. 5.800.992
U.S. Pat. No. 5,807,522
U.S. Pat. No. 5,830,645
U.S. Pat. No. 5,837,196
U.S. Pat. No. 5,838,002
U.S. Pat. No. 5,840,873
U.S. Pat. No. 5,843,640
U.S. Pat. No. 5,843,650
U.S. Pat. No. 5,843,651
U.S. Pat. No. 5.843.663
U.S. Pat. No. 5,846,708
U.S. Pat. No. 5,846,709
U.S. Pat. No. 5,846,717
U.S. Pat. No. 5,846,726
U.S. Pat. No. 5,846,729
U.S. Pat. No. 5,846,783
U.S. Pat. No. 5,846,945
U.S. Pat. No. 5,847,219
U.S. Pat. No. 5,849,481
U.S. Pat. No. 5,849,486
U.S. Pat. No. 5,849,487
U.S. Pat. No. 5,849,497
U.S. Pat. No. 5,849,546
U.S. Pat. No. 5,849,547
U.S. Pat. No. 5,851,772
U.S. Pat. No. 5,853,990
U.S. Pat. No. 5,853,992
U.S. Pat. No. 5,853,993
U.S. Pat. No. 5,856,092
U.S. Pat. No. 5,858,652
U.S. Pat. No. 5.861.244
U.S. Pat. No. 5,863,732
U.S. Pat. No. 5,863,753
U.S. Pat. No. 5,866,331
U.S. Pat. No. 5,866,366
U.S. Pat. No. 5,871,928
U.S. Pat. No. 5,871,986
U.S. Pat. No. 5,876,932
U.S. Pat. No. 5,882,864
U.S. Pat. No. 5.900.481
U.S. Pat. No. 5.905.024
U.S. Pat. No. 5,910,407
U.S. Pat. No. 5,912,124
U.S. Pat. No. 5,912,145
U.S. Pat. No. 5,912,148
U.S. Pat. No. 5,916,776
U.S. Pat. No. 5,916,779
U.S. Pat. No. 5,919,626
U.S. Pat. No. 5,919,630
U.S. Pat. No. 5,922,574
U,S. Pat. No. 5,925,517
U.S. Pat. No. 5,928,862
U.S. Pat. No. 5,928,869
U.S. Pat. No. 5,928,905
U.S. Pat. No. 5,928,906
U.S. Pat. No. 5,929,227
U.S. Pat. No. 5,932,413
U.S. Pat. No. 5,932,451
U.S. Pat. No. 5,935,791
U.S. Pat. No. 5,935,825
U.S. Pat. No. 5,939,291
U.S. Pat. No. 5,942,391
U.S. Pat. No. 5,986,258
U.S. Pat. No. 6,004,755
U.S. Pat. No. 6,087,102
U.S. Pat. No. 6,368,799
U.S. Pat. No. 6,383,749
U.S. Pat. No. 6,617,112
U.S. Pat. No. 6,638,717
U.S. Pat. No. 6,720,138
U.S. Pat. RE 35,413
U.S. Patent Publn. 2008/0009439
Bahr et al., J. Mass Spectrom., 32:1111-1116, 1997.
Bellus, J. Macromol. Sci. Pure Appl. Chem., A31(1): 1355-1376, 1994.
Bentzley et al., Anal Chem., 68(13):2141-2146, 1996.
Bucknall et al., J. Am. Soc. Mass Spectrom., 13(9):1015-1027, 2002.
Cancer Facts and Figures. Atlanta: American Cancer Society; 2015.
Cancer Facts and Figures. Atlanta: American Cancer Society; 2012.
Caprioli et al., Anal. Chem., 69:4751, 1997.
Chaurand et al., Anal Chem., 71)23:5263-5270, 1990.
Chung et al., J. Clin. Oncol.. 24:4170-6, 2006.
Chen et al., Nat Biotechnol., 19:537-542, 2001,
De Jager et al., Semin Nucl Med 23(2) :165-179, 1993.
Desiderio et al., J. Mass Spectrom., 35(6):725-733, 2000.
Doolittle et al., Methods Mol Biol., 109:215-237, 1999.
Duyster et al., Oncogene, 20:5623-37, 2001.
Duncan et al., Rapid Commun. Mass Spectrom., 7(12):1090-1094, 1993.
Faulstich et al., Anal. Chem., 69(21):4349-4353, 1997.
Fenn et al., Science, 246(4926):64-71, 1989.
Fodor et al., Science, 251:767-773, 1991.
Frohman In: PCR Protocols: A Guide To Methods And Applications, Academic Press, N.Y., 1990.
Gobom et al., Anal. Chem 72(14):3320-3326, 2000.
Grzelinski et al., Neoplasia 11:145-56, 2009.
Gulbis and Galand, Human Pathol. 24(12):1271-1285, 1993.
Hacia et al., Nature Genet., 14:441-449, 1996.
Horak et al., Rapid. Commun. Mass Spectrom., 15(4):241-248, 2001.
Horn & Pao, J. Clin. Oncol. 2009; 27:4232-5
Hoshika et al., Nucleic Acids Res., 32:3815-3825, 2004.
Huang et al. Carcinogenesis 34(8): 1833-42, 2013.
Innis et al., Proc. Nat'l Acad. Sci. USA, 85(24):9436-9440, 1988
Jensen et al., BMC Med imaging, 8:16, 2008.
Jeske et al., J. Neurosci., 31:8681-8, 2011.
Jespersen et al., Anal Chem., 71(3):660-666, 1999.
Jiang et al., J. Agric. Food Chem., 48:3305, 2000.
Kanazawa et al., Biol. Pharm. Bull., 22(4):339-346, 1999.
Kashani-Sabet et al., Antisense Res. Dev., 2:3, 1992.
Katayama et al., Sci. Transl. Med. 4. 120ra117, 2012.
Katayama et al., Proc. Nat'l Acad. Sci. USA, 108:7535-40, 2011.
Kazmaier et al., Anesthesiology, 89(4):831-817, 1998.
Knowles et al., Laryngoscope 121:2359-65, 2011.
Koivunen et al., Clin. Cancer Res., 14:4275-83, 2008.
Kwoh et al., Proc. Nat'l Acad. Sci. USA, 86:1173, 1989.
Li et al., Trends Biotechnol., 18:151, 2000.
Li et al., Oral Surg. Oral Med. Oral Pathol. Oral Radiol, 116: 591-7, 201.
Lindeman et al., J. Mol. Diag. [INSERT] 2013.
Lovelace et al., J. Chromatogr., 562(1-2):573-584, 1991.
Fukuoka et al., J. Clin. Oncol., 29:2866-74, 2011.
Lynn et al., J. Mol. Evol., 48(5):605-614, 1999.
Marie et al., Anal. Chem., 72(20):5106-5114, 2000.
Miketova et al., Mol. Biotechnol., 8(3):249-253, 1997.
Mirgorodskaya et al., Rapid Commun. Mass Spectrom., 14(14):1226-1232, 2000.
Miyake et al., Oncogene 2002; 21:5823-34,.
Moon et al., Exp. Biol. Med. (Maywood) 235:907-20, 2010.
Muddiinan et al., Fres. J. Anal. Chem., 354:103, 1996.
Mustafa et al., Indian J. Pharmacol., 47:649-56, 2015.
Nakamura et al., In: Handbook of Experimental Immunology (4th Ed.),'Weir et al, (eds). 1;27, Blackwell Scientific Publ., Oxford, 1987.
Nelson et al., Anal. Chem., 66:1408, 1994.
Nguyen et al. J. Chromatogr. A., 705(1):21-45, 1995.
Ohara et al., Proc. Nat'l Acad. USA, 86:5673-5677, 1989.
Pal & Mandal, Acta Pharmacol Sin, 33:1441-58, 2012.
Pease et al, Proc. Nat Read Sci. USA, 91:5022-5026, 1994.
Petrelli et al, Oral Oncol 50(11): 1041-48, 2014.
Powers et al., J. Biol. Chem. 2002; 277:14153-8.
Roepstorff, EXS, 88:81-97, 2000.
Saghafi et al., Neurosci. Lett., 488:247-51, 2011.
Sambrook and Russell, Molecular Cloning: A Laboratory Manual 3rd Ed., Cold Spring Harbor Laboratory Press, 2001.
Shao et al., Ai Zheng, 21:58-62, 2002.
Shoemaker et al., Nature Genetics, 14:450-456.1996.
Siddiqa et al., J. Oncol., 806382, 2012.
Stoeckli et al., Nature Med., 7(4):493-496, 2001
Szabo et al., Oral Oncol., 47:487-96, 2011.
Takach et al., J. Protein Chem., 16:363, 1997.
Tanic et al., Breast Cancer Res Treatment., 2011.
Tsien et al., Head Neck, 35(9): 1323-30, 2012,
Villanueva et al., Enzyme Microb. Technol. 29:99, 1999.
Walker et al., Proc. Natl. Acad. Sei. USA, 89:392-396, 1992.
Wang et al., Agric. Food. Chem., 47:1549, 1999.
Wang et al,. Neoplasia 13:704-15, 2011.
Wang & Hung, Cell Biosci., 2:13, 2012,
Webb et al., Expert Rev. Anticancer Ther. 2009; 9:331-56.
Welistein, Front. Oncol. 2:192, 2012.
Wittman et al., Biotechnol. Bioeng., 72:642, 2001.
Wu et al., Anal. Chem., 70:456A, 1998.
Wu et al., Biochem. Biophys. Res. Common., 233(1):221-226, 1997.
Wu et al., Biochim. Biophys. Acta, 1466:315-327, 2000
Yang et al., J. Agric. Food. Chem., 48:3990, 2000.
Yu et al., Cancer Epidemol Biomark Prev. 16:553-8. 2007.
Thong et al., Clin. Chen. ACTA., 313:147, 2001.
Zweigenbaum et al., Anal. Chem., 71(13):2294-300, 1999.
Zweigenbaum et al., Pharm. Biomed. Anal., 23(4):723-733, 2000.

1. A method of detecting assessing risk of progression, tissue invasion and/or metastasis in a subject having a squamous cell carcinoma comprising assessing, in a cell containing sample from said subject, the methylation status of one or more of the following promoter regions, or the expression of one or more of the following genes:

| | |
|---|---|
| ALK | Anaplastic Lymphoma Kinase |
| HIF3A | Hypoxia Inducible Factor 3 Alpha |
| TRAF7 | TNF Receptor Associated Factor 7 |
| PTK6 | Protein Tyrosine Kinase 6 |
| TNFRSF10C | Tumor Necrosis Factor Receptor Superfamily |
| | Member 10C |
| NCAM1 | Neural Cell Adhesion Molecule 1 |
| CHL1 | Neural Cell Adhesion Molecule L1-like Protein |
| F7 | Coagulation Factor 7 |
| CCDC92 | Coiled-Coil Domain Containing 92 |
| SLC9A3 | Solute Carrier Family 9 Member 3 |
| IRS4 | Insulin Receptor Substrate 4 |

wherein hypomethylation or overexpression, compared to a subject with stable, non-invasive and or non-metastatic squamous cell carcinoma, of one or more said promoters indicates greater than average risk of progression and/or metastasis.

2. The method of claim 1, wherein ALK, IRS4, and PTK6 are assessed for hypomethylation and/or overexpression, optionally further including assessing EGFR overexpression.

3. The method of claim 1, wherein said cell containing sample is saliva.

4. The method of claim 1, wherein 2, 3, 4, 5, 6, 7, 8, 9 10 or all 11 of said promoter regions are assessed.

5. The method of claim 1, wherein methylation is measured by pyrosequecing or whole genome bisulfite conversion/amplification followed by targeted next generation sequencing or pyrosequencing, and/or wherein expression is measured by FlexMap-based branched DNA probes, microfluidic PCR or droplet digital PCR.

6. The method of claim 1, wherein said subject has oral squamous cell carcinoma or head & neck squamous cell carcinoma.

7. The method of claims 1, wherein said subject is a human.

8. The method of claim 1, wherein said subject is a non-human mammal.

9. The method of claim 1, wherein assessing is repeated a second time.

10. The method of claim 1, wherein squamous cell carcinoma is early stage, late stage, metastatic, recurrent and/or drug resistant.

11. The method of claim 1, further comprising treating said subject with an ALK inhibitor.

12. The method of claim 11, further comprising treating said subject with a second anti-cancer therapy.

13. The method of claim 12, wherein said second anti-cancer therapy is a chemotherapy, a radiotherapy, an immunotherapy, a toxin therapy and/or surgery.

14. The method of claim 12, wherein said second anti-cancer therapy is an anti-EGFR therapy.

15. The method of claim 14, wherein said anti-EGFR therapy is gefitnib, erlotinib, lapatinib, cetuximab, panitumumab, vandetanib, necitumumab, or osimertinib.

16. The method of claim 11, wherein treating comprises administration to a tumor site, or local or regional to a tumor site.

17. The method of claim 11, wherein treating comprises systemic administration.

18. The method of claim 14, wherein treating comprises multiple administrations of said anti-EGFR therapy.

19. The method of claim 11, wherein treating comprises multiple administrations of said ALK inhibitor.

20. The method of claim 1, wherein patients are stratified based on salivary DNA methylation/RNA expression signatures for personalized targeted therapy against ALK and in combination with EGFR targeted drugs.
